(19) United States
(12) Patent Application Publication
(10) Pub. No.: US 2007/0225363 A1
(43) Pub. Date: Sep. 27, 2007

(54) Methods and Agents for Inhibiting Dynamin-Dependent Endocytosis

(75) Inventors: Adam McCluskey, Mayfield (AU); Phillip J. Robinson, North Rocks (AU); Timothy A. Hill, Cooks Hill (AU)

Correspondence Address: EDWARDS ANGELL PALMER & DODGE LLP, P.O. BOX 55874, BOSTON, MA, 02205, US

(73) Assignees: the University of Newcastle Research Associates Ltd., New South Wales (AU); Children's Medical Research Instiute, New South Wales (AU)

(21) Appl. No.: 10/580,098

(22) PCT Filed: Nov. 22, 2004
(86) PCT No.: PCT/AU04/01624

§ 371 Date: Feb. 5, 2007

(30) Foreign Application Priority Data
Nov. 21, 2003 (AU) 2003906456
Dec. 9, 2003 (AU) 2003906823

(51) Int. Cl. A61K 31/275; C07C 255/00; C07C 255/34; C07C 255/40; C07C 255/41; C12N 5/06; C12N 5/08; C12Q 1/34

(52) U.S. Cl. 514/482; 435/18; 435/375; 558/303; 558/402; 558/404; 558/410

(57) ABSTRACT

There are disclosed methods for inhibiting dynamin-dependent endocytosis in cells comprising treating the cells with an effective amount of a compound of formula (I), or a dimeric tyrphostin, physiologically acceptable salt, or prodrug thereof. Compounds useful in the methods described are also provided. The inhibition of dynamin-dependent endocytosis of cells is applicable to the treatment of epilepsy and neurological disorders and conditions.

### FIELD OF THE INVENTION

The present invention relates to agents for inhibiting dynamin-dependent endocytosis and methods for the prophylaxis or treatment of diseases or conditions mediated by dynamin-dependent endocytosis.

### BACKGROUND OF THE INVENTION

Mammalian cells take up extracellular material and recycle their membranes by endocytosis which involves the formation of numerous membrane vesicles at the plasma membrane. The vesicles occur in different sizes, ranging from large phagosomes, smaller clathrin-coated vesicles to tiny synaptic vesicles (SV). Endocytic mechanisms subserve many cellular functions including the uptake of extracellular nutrients, regulation of cell-surface receptor expression and signalling, antigen presentation and maintenance of synaptic transmission.

Among the various endocytic pathways are two that are biochemically well-characterized. The first is rapid synaptic vesicle endocytosis (SVE) that follows vesicle exocytosis in nerve terminals. SVE is not specifically linked to receptor activation but serves to retrieve empty SVs for later refilling, and requires the activity of the enzyme dynamin I. The second is receptor-mediated endocytosis (RME) which is initiated upon ligand binding to cell surface receptors and occurs via clathrin-coated pits in all cells, including nerve terminals. RME provides the main entry point into cells for plasma membrane components (such as the receptor-ligand complexes and membrane lipids) or for extracellular fluid and involves the action of dynamin II. Both RME and SVE operate together within the same neuron but perform distinct functional roles.

Although they share similar underlying protein machinery, RME and SVE utilise distinct isoforms of the same proteins. Multiple subforms of both RME and SVE exist. For example, internalisation of the epidermal growth factor receptor (EGFR) and transferrin receptors are mediated by RME and are dependent on the activity of dynamin, but only the former is sensitive to tyrosine kinase (TK) inhibitors suggesting distinct biochemical requirements for RME of these two activated receptors. Endocytosis plays multiple roles in human pathological conditions including neuronal disorders and a better understanding of how to control endocytosis is clinically important.

Dynamin is the key enzyme which mediates the final stage of endocytosis (Brodin et al., 2000). As well as dynamin, the molecular mechanisms of endocytosis involve many proteins and lipid cofactors that result in dynamin recruitment and its activation (Cousin and Robinson., 2001). The endocytic proteins act sequentially in endocytosis at stages which fall into at least 4 morphological and biochemical categories although some proteins are involved at more than a single stage in the pathway. These morphological and biological categories are:
1. Nucleation: The presynaptic terminal synaptotagmin on the SV functions as the link between exocytosis and endocytosis by recruiting the AP-2 adaptor protein complex to nucleation points at sites of exocytosis. AP-2 recruits clathrin to form a vesicle coat and then amphiphysin.
2. Invagination: Amphiphysin is a docking molecule that recruits most of the remaining endocytic proteins (dynamin, endophilin and synaptojanin) required for the vesicle to invaginate.
3. Fission: Rings of assembled dynamin, amphiphysin and/or endophilin form as a helical collar around the neck of invaginated vesicles. All three of these proteins are able to self-assemble into rings in vitro. Fission of the vesicle neck leading to release of the vesicle requires the GTPase activity of dynamin. GTP hydrolysis produces sudden expansion of the helix pushing the vesicle from the plasma membrane or alternatively, causes ring constriction. In the Drosophila strain shibire, mutations in dynamin's GTPase domain (that do not block GTP binding but block GTP hydrolysis) allow assembly of dynamin helices yet block SV fission after they form (Koenig and Ikeda., 1989). This discriminates between the GTP binding and GTP hydrolysis steps of dynamin's reaction cycle and indicates that GTP hydrolysis that is, GTPase activity, is the last step prior to vesicle fission. Overexpression of GTPase-defective dynamin mutants inhibits both RME and SVE (Brodin et al., 2000).
4. Uncoating. The SV is uncoated and filled with neurotransmitter before being available for exocytosis.

Accordingly, dynamin is a GTPase enzyme required for the retrieval of synaptic vesicles after exocytosis and functions in endocytosis by stimulated assembly as a helix around the neck of invaginating synaptic vesicles (Brodin et al., 2000; Cousin and Robinson., 2001). Dynamin is also a phosphoprotein and is phosphorylated by protein kinase C (PKC) in vitro and by cyclin-dependent protein kinase (Cdk5) in vivo. It is rapidly dephosphorylated by calcineurin on stimulation of endocytosis by depolarisation and calcium influx, and blocking dephosphorylation prevents endocytosis in nerve terminals. It remains dephosphorylated during endocytosis of most vesicles and is rephosphorylated while endocytosis is completing. Hence, the dephosphorylation of dynamin is unlikely to play a role during endocytosis but is probably a priming step prior to endocytosis.

There are three dynamin genes with dynamin I being expressed in neurons while dynamin II is ubiquitously expressed. Dynamin III is expressed in neurons and is highly abundant in testes. All dynamins have four main domains namely, the GTPase domain, the pleckstrin homology (PH) domain, the GTPase effector domain (GED), and a proline rich domain (PRD).

The GTPase domain has an unusually low affinity for GTP (10-25 μm) and extremely high turnover rates compared with other GTPases. It is required for vesicle fission. The crystal structure of this domain of dynamin from Dictyostelium was recently solved (Niemann et al., 2001). The globular structure contains the G-protein core fold, but the normal six-stranded β-sheet is extended to an eight-stranded one by a unique 55 amino acid insertion.

The pleckstrin homology (PH) domain is both a targeting domain and potentially a GTPase inhibitory module and is essential for endocytosis. Dynamin interacts with lipids via this domain, and dynamin binding to nanotubules containing phosphatidylinositol bisphosphate (PtdIns(4,5)P₂) greatly stimulates GTPase activity (Stowell et al., 1999). The PH domain is not needed for self-assembly or GTPase activity and deleting it (delta-PH dynamin) maximally increases intrinsic GTPase activity.

The GTPase effector domain (GED) controls dynamin-dynamin interactions and dynamin assembly into a tetrameric configuration. About 28-32 tetramers cooperatively self-assemble as a single ring or as a helix around PtdIns(4,5)P₂-containing lipid mixtures. GED accounts for tetramer self-association by binding to the GTPase domain. Mutations in GED affect endocytosis in cells, some decreasing and some (surprisingly) increasing endocytosis. GED acts like a GTPase activator protein to stimulate GTPase activity.

The proline-rich domain (PRD) at dynamin's C-terminus interacts with many SH3 domain-containing proteins and calcineurin, and is the site for in vivo dynamin phosphorylation.

Multiple endocytosis inhibitors and methods for inhibiting endocytosis exist such as cationic amphiphilic drugs (eg., chlorpromazine), concanavalin A, phenylarsine oxide, dansylcadaverine, intracellular potassium depletion, intracellular acidification and decreasing medium temperature to 4° C. Each has poor specificity and limited utility. Nonetheless, their use has contributed to a better understanding of endocytosis. Some have been used to demonstrate that blocking endocytosis has clinical implications for humans (Atwood., 2001).

### SUMMARY OF THE INVENTION

The present invention in one or more embodiments relates to compounds capable of inhibiting the GTPase activity of dynamin, and the use of such compounds to inhibit dynamin-dependent endocytosis. In particular, at least some dimeric tyrphostins have been found to be capable of inhibiting endocytosis mediated by dynamin.

Accordingly, in an aspect of the present invention there is provided a method of inhibiting dynamin-dependent endocytosis in cells, the method comprising treating the cells with an effective amount of a compound of formula I, or a physiologically acceptable salt thereof, wherein:
M-Sp-M′ Formula I

M and M′ are each independently a moiety of formula II and are the same or different, and Sp is a spacer;

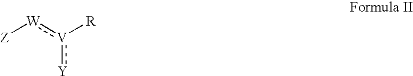

V is C or CH;

W is CH or a linker group; and

Y is hydrogen, cyano, nitro, NH, amino, oxo, halo, hydroxy, sulfhydryl, carboxy, thiocarboxy, sulfur, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur; or

W, V and Y form a 5 or 6 membered substituted or unsubstituted heterocyclic or carbocyclic ring fused with Z, wherein the heterocyclic ring includes from 1 to 3 heteroatoms selected from O, N and S, and the carbocyclic or heterocyclic ring, when substituted, has at least one substituent selected from cyano, nitro, NH, amino, oxo, halo, hydroxy, sulfhydryl, carboxy, thiocarboxy, sulfur, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur; and

R is CH₂R′, CXR′ or CHX′R′;

X is O or S;

X′ is cyano, nitro, amino, halo, hydroxy, sulfhydryl, carboxy, thiocarboxy, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur;

R′ is NH, O or S bonded to the spacer; and

Z is selected from:
(a) an unsubstituted heterocyclic group consisting of one or two rings independently having 5 or 6 ring members including up to 3 heteroatoms selected from O, N and S;
(b) an unsubstituted carbocyclic group consisting of one or two rings independently having 5 or 6 ring members;
(c) a heterocyclic group consisting of one or two rings independently having 5 or 6 ring members including up to 3 heteroatoms selected from O, N and S wherein, the heterocyclic group has one or more substituents independently selected from:
   (i) nitro, NH, amino, cyano, halo, hydroxy, carboxy, oxo, sulfur, sulfhydryl, C₁-C₂ alkoxy and C₁-C₂ acyl; and
   (ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl; and
(d) a carbocyclic group consisting of one or two rings independently having 5 or 6 ring members, and at least two substituents when W is CH or a linker group or W, V and Y form an unsubstituted carbocyclic group, or at least one substituent when W, V and Y form a heterocyclic group, independently selected from:
   (i) nitro, NH, amino, cyano, halo, hydroxy, carboxy, oxo, sulfur, sulfhydryl, C₁-C₂ alkoxy and C₁-C₂ acyl; and
   (ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl; and

wherein when Z of one of M or M′ is selected from (b), Z of the other of M or M′ is selected from (a), (c) or (d).

Preferably, the compound of formula I will be a dimeric tyrphostin.

The invention also relates to the prophylaxis or therapeutic treatment of a disease or condition responsive to inhibition of dynamin-dependent endocytosis. Hence, in another aspect of the present invention there is provided a method of prophylaxis or treatment of a disease or condition in a mammal mediated by dynamin-dependent endocytosis, the method comprising administering to the mammal an effective amount of a compound of formula I, or a physiologically acceptable salt, or prodrug thereof.

In yet another aspect of the present invention there is provided a method of prophylaxis or treatment of a disease or condition in a mammal mediated by dynamin-dependent endocytosis, the method comprising administering to the mammal an effective amount of a dimeric tyrphostin which binds to dynamin and thereby inhibits GTPase activity of the dynamin, or a physiologically acceptable salt, or an analogue, or prodrug thereof.

Treating cells or a mammal with a compound of formula I, or a dimeric tyrphostin or analogue thereof, is to be taken to encompass the administration of compounds that dimerise in vivo to produce a compound of formula I, or a dimeric tyrphostin or analogue thereof which binds to dynamin inhibiting the GTPase activity of the protein, and prodrugs which are processed in vivo to yield or produce a compound of formula I, or dimeric tyrphostin or analogue thereof which binds to dynamin inhibiting the GTPase activity of the protein.

In a further aspect of the present invention there is provided the use of a compound of formula I or a physiologically acceptable salt thereof in the manufacture of a medicament for prophylaxis or treatment of a disease or condition in a mammal mediated by dynamin-dependent endocytosis.

In yet another aspect of the present invention there is provided the use of a dimeric tyrphostin, a physiologically acceptable salt, or an analogue, or prodrug thereof, in the manufacture of a medicament for prophylaxis or treatment of a disease or condition in a mammal mediated by dynamin-dependent endocytosis, wherein the dimeric tysphostin or analogue binds to dynamin inhibiting the GTPase activity of the dynamin.

In still another aspect of the present invention there is provided a compound of formula III or a physiologically acceptable salt thereof, wherein:
M-Sp-M′ Formula III

M and M′ are each independently a moiety of formula IV and are the same or different, and Sp is a spacer.

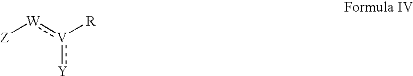

V is C or CH;

W is CH or a linker group; and

Y is hydrogen, cyano, nitro, NH, amino, oxo, halo, hydroxy, sulfhydryl, carboxy, thiocarboxy, sulfur, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur; or

W, V and Y form a 5 or 6 membered substituted or unsubstituted heterocyclic or carbocyclic ring fused with Z, wherein the heterocyclic ring includes from 1 to 3 heteroatoms selected from O, N and S, and the carbocyclic or the heterocyclic ring, when substituted, has at least one substituent selected from cyano, NH, nitro, amino, oxo, halo, hydroxy, sulfhydryl, carboxy, thiocarboxy, sulfur, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur; and

R is CH₂R′, CXR′ or CHX′R′;

X is O or S;

X′ is cyano, nitro, amino, halo, hydroxy, sulfhydryl, carboxy, thiocarboxy, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur;

R′ is NH, O or S bonded to the spacer; and

Z is selected from:
(a) an unsubstituted heterocyclic group consisting of one or two rings independently having 5 or 6 ring members including up to 3 heteroatoms selected from O, N and S;
(b) an unsubstituted carbocyclic group consisting of one or two rings independently having 5 or 6 ring members;
(c) a heterocyclic group consisting of one or two rings independently having 5 or 6 ring members including up to 3 heteroatoms selected from O, N and S, wherein the heterocyclic group has one or more substituents independently selected from:
   (i) nitro, NH, amino, cyano, halo, hydroxy, carboxy, oxo, sulfur, sulfhydryl, C₁-C₂ alkoxy and C₁-C₂ acyl; and
   (ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl; and
(d) a carbocyclic group consisting of one or two rings independently having 5 or 6 ring members, and at least two substituents when W is CH or a linker group or W, V and Y form an unsubstituted carbocyclic group, or at least one substituent when W, V and Y form a heterocyclic group, independently selected from:
   (i) nitro, NH, amino, cyano, halo, hydroxy, carboxy, oxo, sulfur, sulfhydryl, C₁-C₂ alkoxy and C₁-C₂ acyl; and
   (ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl;

wherein when Z of one of M or M′ is selected from (b), Z of the other of M or M′ is selected from (a), (c) or (d), and with the proviso that Z of at least one of M and M′ is other than a benzyl group of formula IVa when R is CXR′, X is O, R′ is NH bonded to the spacer, V is C, W is CH, Y is cyano, and

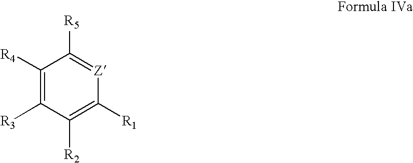

R₁, R₂, and R₅ are H, and R₃ and R₄ are hydroxy; or

R₁ and R₅ are H, and R₂ to R₄ are hydroxy when Sp is a C₂-C₄ alkyl spacer;

wherein Z′ is a carbon atom bonded to W.

Preferably, when the Y substituent of one of M or M′ of a compound of the invention or administered in accordance with the invention is hydrogen, the Y substituent of the other of M or M′ will be other than hydrogen. Typically, Z of at least one of M and M′ will be other than a 2,3-disubstituted carbocyclic group. Preferably, Z of at least one of M and M′ comprises:

at least two substituents in ortho positions relative to one another or in adjacent substitution positions when Z is selected from (d) and W is CH or a C₁-C₃ linker group; or

the, or one of, the substituents on a carbon atom adjacent to the, or one of the, heteroatom(s) when Z is a heterocyclic group selected from (c); or

when W, V and Y are cyclised forming a heterocyclic ring fused with Z, the, or one of, the substituents on a carbon atom of Z spaced at least one bond length from the heterocyclic ring.

In another aspect of the present invention there is provided a prodrug of a compound of formula I or formula III.

In yet another aspect of the present invention there is provided a pharmaceutical composition comprising a compound of formula III, or a physiologically acceptable salt, or prodrug thereof, together with a physiologically acceptable excipient, carrier or diluent.

In still another aspect of the present invention there is provided a method for screening a dimeric tyrphostin or an analogue thereof for ability to bind to dynamin and inhibit GTPase activity of dynamin, the method comprising:

incubating the dimeric tyrphostin or analogue thereof with dynamin or a molecule having dynamin GTPase activity to provide test data; and

determining whether the dimeric typhostin or analogue thereof inhibits the GTPase activity of dynamin on the basis of the test data.

The molecule having dynamin GTPase activity may be a fragment of dynamin that retains GTPase activity or for instance, a homologue, derivative or analogue of dynamin that acts as a substitute for dynamin in the assay.

All publications mentioned in this specification are herein incorporated by reference. Any discussion of documents, acts materials, devices, articles or the like which has been included in the present specification is solely for the purpose of providing a context for the present invention. It is not to be taken as an admission that any or all of these matters form part of the prior art base or were common general knowledge in the field relevant to the present invention as it existed in Australia or elsewhere before the priority date of this application.

Throughout this specification the word “comprise”, or variations such as “comprises” or “comprising” will be understood to imply the inclusion of a stated element, integer or step, or group of elements, integers or steps, but not the exclusion of any other element, integer or step, or group of elements, integers or steps.

The features and advantages of the present invention will become further apparent from the following description of preferred embodiments of the invention together with the accompanying drawings.

### BRIEF DESCRIPTION OF THE FIGURES

FIG. 1: Graphs indicating bis-tyrphostin and tyrphostin A47 (a, b) inhibit the GTPase activity of both dynamin I and dynamin II. The GTPase activity of 0.2 μg dynamin I (c,d) and dynamin II (e, f) was measured using 1.3 Ci[ [symbol] -³²P]-GTP in the presence or absence of bis-tyrphostin (c, e) or tyrphostin A47 (d, f). The basal activity (open circles) and phospholipid-stimulated activity (solid circles) are compared;

FIG. 2: Autoradiograph of nitrocellulose membranes illustrating [α-³²P]GTP binding to dynamin I and dynamin II is not affected by the addition of bis-tyrphostin or tyrphostin A47 (a, b). Quantitative data is shown in panels (c) and (d);

FIG. 3:(a) Graph showing bis-tyrphostin does not act at the PH domain of dynamin I since the compound still inhibits the GTPase activity of a mutant form of recombinant dynamin lacking this domain (“Dynamin I-Delta PH”); (b) photo of an SDS gel stained with Coomassie blue showing that bis-tyrphostin does not block dynamin binding to lipid, with dynamin being retained in the pellet (P) rather than the supernatant (S);

FIG. 4: Fluorimetric assays of exocytosis (a,c) and endocytosis (b,d) in isolated nerve terminals (synaptosomes) shows bis-tyrphostin but not A47 specifically decreases endocytosis. Retrieval efficiency is a more accurate measurement of endocytosis in relation to the preceding amount of exocytosis, and bis-tyrphostin produced a significant block in retrieval efficiency (e).

FIG. 5: Electron micrographs of isolated rat brain nerve terminals (synaptosomes) showing synaptic vesicle depletion in synaptosomes upon addition of bis-tyrphostin followed by stimulation by depolarisation (a, b) and the accumulation of vesicle invaginations and collared pits (c-h); and

FIG. 6: Photographs showing that internalisation of texas-red labelled transferrin into Swiss 3T3 cells (a-d) or HER14 cells (e-h) is inhibited by a 15 minute preincubation with 100 μM bis-tyrphostin. DAPI (blue) staining indicates the cell nuclei.

### DETAILED DESCRIPTION OF PREFERRED EMBODIMENTS OF THE INVENTION

The term “alkyl” as used herein encompasses straight or branched chain saturated aliphatic groups. By “C₁-C₂ alkyl” is meant the length of the alkyl chain. Such alkyl groups include methyl, ethyl, 1-methyl-ethyl and 1,1-dimethyl-ethyl groups.

The term “C₁-C₂ group” or “C₁-C₃ group” as used herein encompasses saturated or unsaturated aliphatic groups of the specified number of carbon atoms in length and which may be branched or unbranched. Such groups include alkyl and alkenyl groups. Alkenyl groups include at least one double bond. Examples of C₁-C₃ groups include methyl, ethyl, propyl, isopropyl, 1,3-dimethylpropyl, 1-methyl-3-ethylpropyl, ethenyl, 1-propenyl, 2-propenyl, 1-methyl-2-propenyl and 2-methyl-1-propenyl.

The term “C₁-C₂” alkenyl group as used herein encompasses C₁ groups linked to a heterocyclic or carbocyclic group of Z by a double bond.

The term “C₁-C₂ alkoxy” as used herein encompasses alkoxy groups of the specified number of carbon atoms in length. The alkoxy group may include a carbon-carbon double bond.

The term “carbocyclic group” as used herein encompasses groups comprising one or more rings of carbon atoms. The ring, or at least one of the rings, of the carbocyclic group may have one or more multiple bonds. The carbocyclic group formed when W, V and Y are cyclised in a compound of formula I or III will preferably include one or more double bonds.

The term “heterocyclic group” as used herein encompasses groups comprising one or more rings of atoms wherein the ring, or at least one of the rings, includes a heteroatom selected from O, N and S. The ring, or at least one of the rings, may also have one or more multiple bonds.

By the term “dimeric tyrphostin” is meant a compound comprising two tyrphostin moieties linked together by a spacer moiety wherein the tyrphostin moieties are the same or different. Typically, each tyrphostin moiety will be the same. Most preferably, each tyrphostin moiety will be a benzylidenemalonitrile moiety. Bis-tyrphostin is one such dimeric tyrphostin which has now surprisingly been found to be capable of binding to dynamin and inhibiting the GTPase activity of the protein.

Preferably, M and M′ of a compound of formula I or III will each independently be a moiety of formula V 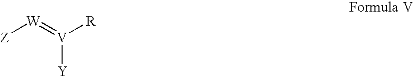
wherein:

V is C;

W is CH;

Y is hydrogen, cyano, nitro, amino, halo, hydroxy, sulfhydryl, carboxy, thiocarboxy, or an unsubstituted C₁-C₂ group or C₁-C₂ group substituted with at least one group independently selected from cyano, nitro, NH, amino, oxo, halo, hydroxy, sulfhydryl, carboxy, thiocarboxy and sulfur; or

W, V and Y form a 5 or 6 membered substituted or unsubstituted heterocyclic or carbocyclic ring fused with Z, wherein the heterocyclic ring includes from 1 to 3 heteroatoms selected from O, N and S, and the carbocyclic or heterocyclic ring, when substituted, has at least one substituent selected from cyano, nitro, NH, amino, oxo, halo, hydroxy, sulfhydryl, carboxy, thiocarboxy and sulfur, or an unsubstituted C₁-C₂ group or C₁-C₂ group substituted with at least one group independently selected from cyano, nitro, NH, amino, oxo, halo, hydroxy, sulfhydryl, carboxy, thiocarboxy and sulfur; and

R is CH₂R′, CXR′ or CHX′R′;

X is O or S;

X′ is cyano, nitro, amino, halo, hydroxy, sulfhydryl, carboxy, thiocarboxy, or an unsubstituted C₁-C₂ group or C₁-C₂ group substituted with at least one group independently selected from cyano, nitro, NH, amino, oxo, halo, hydroxy, sulfhydryl, carboxy, thiocarboxy and sulfur; and

R′ is NH, O or S bonded to the spacer; and

Z is a group as in formula II.

Preferably, when W, V and Y are not cyclised, Y will be cyano, nitro, amino, hydroxy, carboxy or thiocarboxy. Most preferably, Y will be cyano.

Preferably, R is CXR′ wherein X is O or S and R′ is NH, O or S. More preferably, X will be O or S and R′ will be NH.

When Z is a carbocyclic group it may include one or more double bonds. The carbocyclic group may for instance, be a cycloalkanyl group, an aryl group such as a phenyl or naphthyl group, or a polyphenyl group such as bi-phenyl. When the carbocyclic group comprises two rings, the ring bonded directly to W will preferably bear all the substituents, or have at least two substituents when W is CH or linker group or have the, or at least one of, the substituents when W, V and Y are cyclised. Preferably, Z will be a group selected from:
(i) a heterocyclic group consisting of one or two rings independently having 5 or 6 ring members including up to 3 heteroatoms independently selected from O, N and S;
(ii) a heterocyclic group consisting of one or two rings independently having 5 or 6 ring members including up to 3 heteroatoms selected from O, N and S, wherein the heterocyclic group has one or more substituents independently selected from nitro, NH, halo, cyano, amino, hydroxy, carboxy, oxo, sulfur, and C₁-C₂ alkoxy; and
(iii) an carbocyclic group consisting of one or two rings independently having 5 or 6 ring members, and at least two substituents independently selected from nitro, NH, amino, halo, cyano, hydroxy, carboxy, oxo, sulfur and C₁-C₂ alkoxy.

Preferably, when the Z group is a carbocyclic group and has a halo, cyano, C₁-C₂ alkoxy or C₁-C₂ acyl substituent, the Z group will also generally have at least two other substituents, preferably independently selected from nitro, NH, amino, hydroxy, carboxy, oxo and sulfur, and most preferably from nitro, NH, amino, hydroxy and carboxy. Preferably, the carboxycyclic group will be an aryl group and most preferably, a substituted benzyl group.

Preferably, when the Z group is a heterocyclic group it will have one or two rings independently having 5 or 6 ring members including up to 3 heteroatoms selected from O and N, wherein the heterocyclic group has one or more substituents independently selected from nitro, NH, amino, halo, hydroxy, carboxy and oxo, or an aryl group having a single ring of 5 or 6 ring members and at least two substituents independently selected from nitro, amino, halo, hydroxy and carboxy. Preferably, the aryl group will be a substituted phenyl group.

Preferably, the heterocyclic group will be a substituted or unsubstituted imadazolyl, pyranyl, isobenzylfuranyl, furyl, chromenyl, pyrrolyl, 2H-pyrrolyl, pyrazolyl, pyridyl, pyrazinyl, pyrimidinyl, pyridazinyl, indolizinyl, isoindolyl, 3H-indolyl, indolyl, indazolyl, purinyl, quinolizinyl, isoquinolyl, quinolyl, pthalazinyl, naphthyridinyl quinoxalinyl, quinazolinyl, cinnolinyl, pteridinyl, thienyl, or benzothienyl group. Most preferably, the heterocyclic group will be a substituted such group.

In the instance W, V and Y form a 5 or 6 membered heterocyclic ring fused with Z, the resulting group incorporating Z will typically be a substituted or unsubstituted two ring heterocyclic group. The resulting group may for instance be a substituted or unsubstituted heterocyclic group selected from imadazolyl, chromenyl, indolizinyl, isoindolyl, indolyl, indazolyl, purinyl, quinolizinyl, isoquinolyl, quinolyl, pthalazinyl, napthyridinyl, quinoxalinyl, quinazolinyl, cinnolnyl, pteridinyl, benzothienyl and isobenzofuranyl. Preferably, the resulting group will be substituted or unsubstituted chromenyl, indolyl, or isoquinoline. Again, the heterocyclic group formed by the cyclisation of W, V and Y will preferably be a substituted group.

Most preferably, a compound of the invention or administered to a mammal in accordance with a method of the invention will be a compound wherein:

M and M′ are each independently a compound of formula VI and are the same or different, and

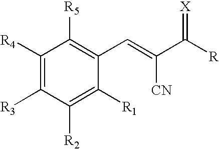

X is O or S;

Y is cyano, nitro, amino, halo, hydroxy, sulfhydryl, carboxy, or thiocarboxy; or

R₁ and Y are cyclised forming a 5 or 6 membered substituted or unsubstituted heterocyclic or carbocyclic ring, wherein the heterocyclic ring includes 1 or 2 heteroatoms selected from O, N and S, and the carbocyclic or heterocyclic ring, when substituted, has at least one substituent selected from cyano, nitro, NH, amino, oxo, halo, hydroxy, sulfhydryl, carboxy, thiocarboxy and sulfur; and

R₂ to R₅ are independently hydrogen or a substituent independently selected from nitro, amino, halo, hydroxy, carboxy, sulfhydryl, thiocarboxy, C₁-C₂ alkoxy and C₁-C₂ acyl; or

R₁ to R₅ are independently hydrogen or a substituent independently selected from nitro, amino, halo, hydroxy, carboxy, sulfhydryl, thiocarboxy, halo, C₁-C₂ alkoxy and C₁-C₂ acyl; and

R is NH, O is S bonded to the spacer Sp;

wherein at least one of M and M′ is characterised in that, at least two of R₁ to R₅ are other than hydrogen and when R₁ to R₂ are other than hydrogen at least one of R₃ to R₅ is also other than hydrogen, or when R₁ and Y are cyclised, at least two of R₂ to R₅ are other than hydrogen when Y and R₁ form an unsubstituted carbocyclic group or at least one of R₂ to R₅ is other than hydrogen when Y and R₁ form a heterocyclic group.

Preferably, when Y and R₁ are not cyclised and R₁ and R₂ are other than hydrogen, R₃ will also be other than hydrogen. Typically, the at least two substituents of R₁ to R₅ will be in an ortho position relative to one another. When the compound has three substituents it is preferred the substituents are adjacent to each other. Preferably, in this instance, either R₁ to R₃ are other than hydrogen or R₂ to R₅ are other than hydrogen.

Typically, when at least one of R₁ to R₅ or R₂ to R₅ is halo, C₁-C₂ alkoxy or C₁-C₂ acyl, there will be at least one other substituent selected from nitro, amino, hydroxy, carboxy and thiocarboxy when R₁ and Y are cyclised and form a heterocyclic ring, or at least two, other substituents selected from those substituents when R₁ and Y are not cyclised or form an unsubstituted carbocyclic ring.

Halo substituents will typically be selected from fluoro, chloro, bromo, and iodo. Preferably, a halo substituent will be selected from fluoro and chloro.

Preferably, the linker group of a moiety of formula I or III will comprise a single atom or a chain of up to three atoms in length wherein the, or one or more of the atoms, may be an atom other than carbon such as N, O or S. Preferably, the linker group will be a C₁-C₃ linker group. The linker group may be substituted or unsubstituted, and may include one or more double bonds. Substituents may for instance be selected from hydroxy, amino, halo, nitro or groups which essentially do not adversely impact on the activity of the compound. Most preferably, the linker group will be unsubstituted.

Preferably, the spacer moiety Sp of a compound of the invention or administered to a mammal in accordance with the invention will permit the compound to adopt a hairpin conformation. Most preferably, the spacer moiety will be a substituted or unsubstituted 1 to 7 atom chain which may include one or more atoms other than carbon such as N, O or S, and one or more double bonds. However, any suitable spacer may be utilised which allows inhibition dynamin-dependent endocytosis by the compound. The spacer may for instance be substituted with one or more groups independently selected from hydroxy, amino, halo and nitro, or other group which does not substantially effect the flexibility or conformation of the chain. Most preferably, the spacer moiety will be a substituted or unsubstituted alkane chain. Typically, the spacer will be an unsubstituted alkane chain having the structure:
—CH₂(CH₂)ₙCH₂—

wherein n is an integer of from 1 to 5 and more usually 2 or 3.

Suitable pharmaceutically acceptable salts include acid and amino acid addition salts, esters and amides that are within a reasonable benefit/risk ratio, pharmacologically effective and appropriate for contact with animal tissues without undue toxicity, irritation or allergic response. Representative salts include hydrochloride, sulfate, bisulfate, maleate, fumarate, succinate, tartrate, tosylate, citrate, lactate, phosphate, oxalate and borate salts. Such salts may for instance be prepared by mixing the corresponding acid with a compound of formula I or dimeric tyrphostin or analogue thereof. The salts may include alkali metal and alkali earth cations such a sodium, calcium, magnesium and potassium, as well as ammonium and amine cations. Suitable pharmaceutical salts are for example exemplified in S. M Berge et al, J. Pharmaceutical Sciences (1997), 66:1-19, the contents of which is incorporated herein in its entirety by cross-reference. Representative esters include C₁-C₇ alkyl, phenyl and phenyl(C₁₋₆) alkyl esters. Preferred esters include methyl esters.

Prodrugs of compounds of formulae I and III, or of dimeric tyrphostins and analogues thereof, include those in which groups selected from carbonates, carbamates, amides and alkyl esters have been covalently linked to free amino, amido, hydroxy or carboxylic groups of the compounds, dimeric tyrphostins and analogues thereof. Suitable prodrugs also include phosphate derivatives such as acids, salts of acids, or esters, joined through a phosphorus-oxygen bond to a free hydroxyl or other appropriate group of a compound of formula I or III, or dimeric tyrphostin or analogue thereof. A prodrug may for example be inactive when administered but undergo in vivo modification into the active compound that binds to dynamin such that the GTPase activity of the protein is inhibited, as a result of cleavage or hydrolysis of bonds or other form of bond modification post administration. Preferably, the prodrug form of the active compound will have greater cell membrane permeability than the active compound thereby enhancing potency of the active compound. A prodrug may also be designed to minimise premature in vivo hydrolysis of the prodrug external of the cell such that the cell membrane permeability characteristics of the prodrug are maintained for optimum availability to cells and for systemic use of the compound.

Endocytosis is a major contributor or direct cause of diverse human diseases. A list of vesicle trafficking-specific diseases has been published, see for example Aridor and Hannan 2000, Traffic 1:836-851 and Aridor and Hannan 2002, 3:781-790 the contents of which are incorporated herein by reference in their entirety. Accordingly, methods of the invention may for instance be useful in the prophylaxis or treatment of cancers, ophthalmologic disease, immunodeficiency diseases, gastrointestinal diseases, viral and bacterial infections, other pathogenic infections, neurodegenerative, neurological and kidney diseases and conditions, and other disorders which involve dynamin-dependent endocytosis, or which are otherwise sensitive to inhibition of dynamin-dependent endocytosis.

For example, it is known that human polyomavirus JCV is the etiologic agent of progressive multifocal leukoencephalopathy, a fatal central nervous system (CNS) demyelinating disease and its entry to neurons is blocked by endocytosis inhibitors such as chlorpromazine (Atwood W., 2001). Similarly, infection by HIV (Wyss S. et al., 2001), influenza virus (Roy A., et al. 2000) and adeno-associated virus (Duan D. et al., 1999) is by endocytosis or is sensitive to its inhibitors.

In addition, growth factor receptors (e.g. EGF-R) require dynamin for internalisation and maintenance of cellular activities from signalling to cell growth (Seto E. et al., 2002). Blocking endocytosis with dynamin constructs prevents cell proliferation in many of these examples (Grieb T. et al., 2000) and provides evidence that dynamin II (the non-neuronal form) inhibitors may have anti-cancer activity. Dent's disease (polycystic kidney disease) also involves endocytosis of ClC-5 chloride channel and endocytosis blockers prevent its internalisation (Schwake M. et al., 2001).

Dynamin is central to all endocytic trafficking from the cell surface, the Golgi apparatus, endosomes and mitochondria. Several neurodegenerative diseases are associated with these trafficking pathways. Two are implicated in generation of β-amyloid, namely the endocytic and the secretory pathways (Aridor & Hannan 2000). In the brain, disease and conditions in which endocytosis plays a role include Alzheimer's disease, Huntington's disease (HD), stiff-person syndrome, Lewy body dimentias, and Niemann-Pick type C disease (Cateldo et al., 2001; Metzler et al, 2001; Ong et al., 2001; Smith et al., 2000).

In Alzheimer's disease β-amyloid precursor protein (APP) is internalized from axonal cell surfaces in clathrin-coated vesicles and sorted away from recycling synaptic vesicles, and transported to endosomes and the cell soma (Marquez-Sterling N. et al., 1997). The endosome is the first compartment along the dynamin-dependent endocytic pathway after internalization of APP or ApoE (Smythies J., 2000) and endosomal alterations are evident in pyramidal neurons in Alzheimer brain (Cataldo A. et al., 1997). Endocytic pathway activation is prominent in APP processing and β-amyloid formation and is an early feature of neurons in vulnerable regions of the brain in sporadic Alzheimer's disease (Cataldo A. et al., 2001).

Huntington's disease (HD) is a neurodegenerative disorder principally affecting striatal neurons, yet the mutated gene product huntingtin is not brain-specific. Huntingtin interacts strongly with members of the Huntingtin-interacting protein 1 (HIP1) family. The huntingtin-HIP1 interaction is restricted to the brain and is inversely correlated to the polyglutamine length in huntingtin. Loss of normal huntingtin-HIP1 interaction may contribute to a defect in membrane-cytoskeletal integrity in the brain. HIP1 is a fundamental component of the dynamin-mediated endocytic machinery (Metzler M. et al., 2001). Hence, numerous reports have linked the neurological defects in HD to endocytosis abnormalities (Aridor & Hannan, 2000; Metzler M. et al., 2001).).

Another example is the presynaptic synuclein protein which is a prime candidate for contributing to Lewy body diseases, including Parkinson's disease, Lewy body dementia and a Lewy body variant of AD. Exogenous synuclein causes neuronal cell death due to its endocytosis and formation of intracytoplasmic inclusions. Cell death and α-synuclein aggregates are direct consequences of its endocytosis in human neuroblastoma cells (Sung J. et al., 2001). Endocytosis has also been implicated in epilepsy. For example, mice with targeted disruption of either of two endocytic proteins synaptojanin (SJ) or amphiphysin have reduced SVE and die from random seizures throughout their lives (Di Paolo et al., 2002) indicating a role in neuronal excitability and a link to epilepsy.

Endocytic pathways are also utilized by viruses, toxins and symbiotic microorganisms to gain entry into cells. For instance, botulism neurotoxins and tetanus neurotoxin are bacterial proteins that inhibit transmitter release at distinct synapses and cause two severe neuroparalytic diseases, tetanus and botulism. Their action is dependent on their internalisation via endocytosis into nerve terminals (Humeau et al., 2000). Hence targeting endocytosis with inhibitors has application as a clinically useful strategy.

Accordingly, examples of specific diseases and conditions for which methods of the invention may be useful for the prophylaxis or treatment of include but are not limited to, multifocal leukoencephalopathy, polycystic kidney disease, β-amyloid associated diseases, Alzheimer's disease, Huntington's disease, stiff-person syndrome, Lewy body diseases, Lewy body dimentias, Parkinson's disease, epilepsy, tetanus, botulism, HIV infection, influenza and mucolipidosis.

Preferably, the compound of formula I administered to a mammal in accordance with the invention will be a dimeric benzylidenemalonitrile tyrphostin or prodrug thereof. Most preferably, the dimeric tyrophostin will be bis-tyrphostin or an analogue thereof. With knowledge of the features and/or groups of bis-tyrphostin or dimeric tyrphostin that provide the ability to bind to and inhibit the activity of dynamin, analogues and more particularly mimetics may be designed that while differing in structure nevertheless retain this capacity. The use of dimeric tyrphostin analogues and particularly analogues of bis-tyrphostin in methods described herein is expressly encompassed by the present invention.

The term “analogue” encompasses a molecule that differs from the dimeric tyrphostin but retains similarity in one or more features that provide the biological function or activity characteristic of the dimeric tyrphostin. An analogue may have substantial overall structural similarity with the dimeric tyrphostin or only structural similarity with one or more regions of the dimeric tyrphostin responsible for the provision of the biological function or activity, or which otherwise have involvement in the provision of the biological function or activity. An analogue of bis-tyrphostin may for instance be provided by substituting one or both hydroxy substituents on one or both aromatic groups of the compound with another suitable group or a number of different suitable groups as described above. Alternatively, or as well, one or more other groups of the compound may be removed, modified or replaced.

The design of an analogue typically involves determining the physical properties of the original compound such as size, charge distribution and tertiary structure and/or identifying which features of the compound are necessary for retaining the capacity to bind to dynamin. In particular, the original compound may be modelled, taking into account the stereochemistry and physical properties of the compound utilising x-ray chrystallography, nuclear magnetic resonance and commercially available computer modelling software. In a preferred variation of this approach, the modelling will take into account the interaction of the compound with dynamin itself such that any change in conformation arising from the interaction may be considered in the design of the analogue. Such modelling techniques are well known in the art and are well within the scope of the skilled addressee. Suitable modelling approaches include the use of Accelrys Catalyst® Pharmacore Development and Accelrys Cerius 4.8 LigandFit® protocols (Accelrys Inc., San Diego, Calif., USA). Further suitable modelling approaches include the use of MacSpartan Pro Version 1.1 protocols (Wave Function Inc, Irvine, Calif., USA).

The provision of an analogue can also involve selecting or deriving a template molecule onto which chemical groups are added to provide the required physical and chemical characteristics, or for facilitating further chemical reactions for obtaining the required physical and chemical characteristics. The selection of template molecule and chemical groups is based on ease of synthesis, risk of potential for degradation in vivo, stability and maintenance of biological activity upon administration. Pharmacological acceptability and the like are also taken into consideration in the design as is understood by the skilled addressee.

Compounds may be administered in accordance with the invention with one or more other compounds or drugs. For example, a compound may be co-administered to the subject mammal in combination or in conjunction with chemotherapeutic drugs or drugs conventionally used in the prophylaxis or therapeutic treatment of the particular disease or condition for which the mammal is being treated. By “co-administered” is meant simultaneous administration in the same formulation or in two different formulations by the same or different routes, or sequential administration by the same or different routes. By “sequential” administration is meant administration one after the other which may involve a time delay between administration of the compound and the other drug or drugs ranging from very short periods up to hours or days.

Suitable pharmaceutical compositions include solutions suitable for injection. Such injectable compositions will be fluid to the extent that syringablity exists and typically, will be stable for at least several months to allow for storage after manufacture. The carrier may be a solvent or dispersion medium containing one or more of surfactants, physiological saline, ethanol, polylol, (e.g. glycerol, propylene glycol, liquid polyethylene glycol and the like), vegetable oils, and mixtures thereof.

For oral administration, the compound may be formulated with an orally acceptable inert diluent, an assimilable edible carrier or it may for instance, be enclosed in a hard or soft shell gelatin capsule. Alternatively, it may be added directly to food. Moreover, the compound may be incorporated with one or more excipients such as dicalcium phosphate, a disintegrating agent such as corn starch, potato starch, or alginic acid and used in the form of ingestible tablets, buccal tablets, troches, capsules, elixirs, suspensions and syrups.

Tablets, pills and the like may also contain one or more of a binder such as gum tragacanth, acacia, corn starch or gelatin, a lubricant such as magnesium stearate, a sweetening agent such as sucrose, lactose, saccharin, and a flavouring agent. When the dosage form is a capsule, it may contain a liquid carrier in addition to one or more of the above ingredients. Various other ingredients may be present as coatings. In addition, the compound may be incorporated into any suitable sustained release preparation or formulation.

The compound will typically be formulated into a pharmaceutical composition with a pharmaceutically acceptable carrier or excipient for administration to the intended subject.

Any conventionally known such carriers diluents and excipients deemed suitable may be used. Suitable pharmaceutically acceptable carriers and excipients include any known appropriate solvents, dispersion media and isotonic preparations or solutions. Use of such ingredients and media for pharmaceutically active substances is well known. Typically, a composition of the invention will also incorporate one or more preservatives such as parabens, chlorobutanol, phenol, sorbic acid, and thimersal. Suitable pharmaceutically acceptable carriers and formulations useful in compositions of the present invention are for instance described in handbooks and texts well known to the skilled addressee, such as “Remington: The Science and Practice of Pharmacy (Mack Publishing Co., 1995)”, the contents of which is incorporated herein in its entirety by reference.

It is particularly preferred to formulate parenteral compositions in dosage unit form for ease of administration and uniformity of dosage. Dosage unit form as used herein is to be taken to mean physically discreet units suited as unitary dosages for the subject to be treated, each unit containing a predetermined quantity of active agent calculated to produce the desired prophylactic or therapeutic effect in association with the carrier and/or excipient selected.

The dosage of the compound to be administered will depend on a number of factors including whether the compound is to be administered for prophylactic or therapeutic use, the condition for which the agent is intended to be administered, the severity of the condition, the age of the subject, and related factors such as weight and general health of the subject as may be determined by the physician or medical attendant in accordance with accepted principles. For example, a low dosage may initially be given which is subsequently increased following evaluation of the subject's response. Similarly, frequency of administration may be determined in the same way that is, by continuously monitoring the subject's response between each dosage and if necessary, increasing the frequency of administration or alternatively, reducing the frequency of administration.

The route of administration of a pharmaceutical composition will again depend on the nature of the disease or condition for which the composition is to be administered. Suitable routes of administration may include but are not limited to respiratoraly, intratrachealy, nasopharyngealy, intravenously, intraperitonealy, subcutaneously, intraderamaly, intramuscularly, by infusion, orally, rectally, topically and by slow-release implant. In the case of intravenous routes, particularly suitable routes are via injection into blood vessels which supply a tumour or particular organs to be treated. Compounds may also be delivered into cavities such as for example the pleural or peritoneal cavity, or be injected directly into tumour or afflicted tissue.

In order that the nature of the present invention may be more clearly understood, preferred forms thereof will now be described with reference to the following non-limiting examples.

### EXAMPLE 1

### Identification of Tyrphostin Inhibitors of Dynamin GTPase Activity

1.1 Materials and Assays Phosphatidylserine, 1,2-diolein, calmodulin, ATP, GTP, leupeptin, phenylmethylsulfonylfluoride, Tween 80, bis(sulfosuccinimidyl)suberate (BS3) and glutathione agarose were obtained from Sigma. Papain and antipain-dihydrochloride were obtained from Boehringer Mannheim (Federal Republic of Germany). Gel electrophoresis reagents and equipment were sourced from Bio-Rad. [ [symbol] -³²P]ATP (3000 Ci/mmol) and [ [symbol] -³²P]GTP (25 μCi/mmol) were from Amersham plc, UK. Protein molecular weight markers and chromatography resins were, sourced from Pharmada. All other reagents were of analytical reagent grade or better.

1.1.1 Production of Proteins

The plasmid for GST-Amph2-SH3 (muscle Amph2) (Butler et al., 1997) was provided by Pieto DeCamilli, Yale, Connecticut, USA, in pGEX2T vectors. The plasmid was grown in E. coli and the GST-Amph2-SH3 fusion protein was purified on glutathione (GSH)-Sepharose by elution with 10 mM reduced GSH in 20 mM Tris-HCl, pH 7.5, dialysed against the same buffer without GSH and stored at 4° C. Dynamin was purified from sheep brain by extraction from the peripheral membrane fraction of whole brain (Robinson et al., 1993) and affinity purification on GST-Amph2-SH3-sepharose as previously described (Marks and McMahon., 1998), yielding 8 mg protein from 250 g sheep brain. Recombinant dynamin II was expressed in insect cells and was a gift from Dr Sandra Schmid (Scripps, San Diego, Calif.). Recombinant dynamin I lacking the PH domain (dynamin PH, provided by Robin Scaife) was expressed in insect cells using baculoviral infection (Salim et al., 1996).

1.1.2 GTPase Assay

Dynamin GTPase activity was determined by hydrolysis of [ [symbol] -³²P]GTP by a method modified from that described previously (Robinson et al., 1993). Briefly, purified dynamin I or dynamin II (0.2 μg/tube) was incubated in GTPase buffer (10 mM Tris, 10 mM NaCl, 2 mM Mg²⁺, 0.05% Tween 80, pH 7.4, 1 μg/ml leupeptin and 0.1 mM PMSF) and a GTP cocktail containing 0.3 mM GTP and 1.3 μCi [ [symbol] -³²P]-GTP in the presence or absence of varying concentrations of inhibitors or DMSO vehicle for 10 min at 30 C. The final assay volume was 40 μl. Dynamin activity was measured as either basal or phospholipid-stimulated with the addition of 5 μg/ml L-phosphatidylserine. The reaction was terminated with 100 μl of GTPase stop buffer (2% formic add, 8% acetic acid, pH 1.9), followed by 600 μl of acid-washed charcoal solution (7% charcoal in acidic solution (w/v)) and 100 μl BSA (5 mg/ml). After centrifuging for 5 min (13,000 rpm at room temperature), 200 μl of each supernatant was counted in a -counter for the release of ³²Pᵢ from [ [symbol] -³²P]-GTP.

1.1.3 [α-³²P]-GTP Binding Assay

The [α-³²P]-GTP-binding assay was performed in the wells of a 96-well microtitre plate. Dynamin (0.2 μg/well) was added to GTPase buffer and incubated for 10 min at 4 C in the dark. [α-³²P]-GTP (2 μCi/tube) was then added to the reaction and incubated for a further 10 min at 4 C. in the dark. The microtitre plate was then irradiated with a short wavelength ultraviolet lamp at 315 nm for 30 min at a distance of 8 cm. The specificity of photolabelling was determined by comparing the labelling in the presence and absence of 1 mM cold GTP. Samples were then applied to nitrocellulose membranes by aspiration through the wells of a 24 well slot blotter. The nitrocellulose was washed 3 times with PBS and dried. Bound nucleotide was detected by a phosphorimager (Molecular Dynamics).

1.1.4 Phospholipid Binding and Helix Assembly

Dynamin I (50 μg/ml) purified from whole sheep brain was incubated with phosphatidylserine liposomes (80 μg/ml, sonicated into 30 mM Tris/HCl pH 7.4) in 100 μl of assembly buffer (1 mM EGTA, 30 mM Tris, 100 mM NaCl, 1 mM DTT, 1 mM PMSF, and Complete protease inhibitor cocktail tablet (Roche)) in the presence or absence of 1 mM Mg/GTP for 1 hour at 25° C. The samples were centrifuged at 14,000 rpm for 15 min to separate lipid-bound (P) and free (S) dynamin and the fractions analysed by gel electrophoresis on a 12% SDS polyacrylamide gel. When present, drugs (10 μM and 100 μM) were pre-mixed with the phospholipid before incubating with dynamin I.

1.1.5 Texas Red-transferrin Uptake in Cells

Transferrin (Tf) uptake was analysed in Swiss 3T3 and HER14 cells based on methods previously described (van der Bliek et al., 1993). Briefly, cells were plated to 60% confluency in DMEM medium plus 10% foetal calf serum after which the cells were incubated overnight (8-10 hours) in DMEM minus foetal calf serum. Texas red-transferrin (Tf-TxR, Molecular Probes, Oregon) was added to a final concentration of 5 μg/ml and the cells incubated at 37° C. for 10 minutes. Cell surface staining was removed by incubating the cells in an ice cold acid wash solution (0.2 M acetic acid +0.5 M NaCl, pH 2.8) for 15 minutes. Cells were immediately fixed with 4% paraformaldehyde for 10 minutes then washed 3 times with PBS. Nuclei were stained using DAPI (Molecular Probes, Oregon). Slides were mounted using DABCO and the fluorescence was monitored using a Leica DMLB bright field microscope and SPOT digital camera. In experiments with inhibitors, the DMEM was supplemented with bis-tyrphostin 15 or 60 minutes before the addition of Tf-TxR.

1.1.6 Endocytosis

Isolated nerve terminals (synaptosomes) were prepared from rat cerebral cortex by centrifugation on discontinuous percoll gradients (Dunkley et al., 1986). Fractions 3 and 4 were pooled and used in all experiments. Endocytosis was measured using uptake of the fluorescent dye FM2-10 as previously described (Cousin and Robinson 2000a). Synaptosomes (0.6 mg in 2 ml) were incubated for 5 min at 37° C. in plus or minus Ca²⁺ Krebs-like solution. FM2-10 (100 M) was added 1 min before stimulation with 30 mM KCl (S1). As FM2-10 is taken up by vesicles via endocytosis at the S1 phase of stimulation, synaptosomes were incubated with antagonists during this phase. Specifically, synaptosomes were incubated with tyrphostin A47 or bis-tyrphostin for 5 min prior to stimulation. After 2 min of stimulation synaptosomes were washed twice in plus Ca²⁺ solution containing 1 mg/ml bovine serum albumin. The washing steps remove non-internalised FM2-10 and the tyrphostins. Washed synaptosomes were resuspended in plus Ca²⁺ solution at 37° C., transferred to a fluorimeter cuvette and stimulated with a standard addition of 30 mM KCl (S2). The standard S2 stimulation releases all accumulated FM2-10 and allows endocytosis to be measured as the decrease in FM2-10 fluorescence due to dye release into solution (excitation 488 nm, emission 540 nm).

Endocytosis was calculated as the decrease in absolute fluorescence stimulated by 30 mM KCl at S2. The displayed traces represent the average release of FM2-10 from synaptosomes after subtraction of background traces acquired from synaptosomes loaded with FM2-10 in the absence of Ca²⁺. Retrieval efficiency is a more accurate measure of endocytosis since it takes into account the amount of prior exocytosis. Retrieval efficiency was calculated as endocytosis/exocytosis, where endocytosis is defined as above and exocytosis as Ca²⁺-dependent glutamate release after 2 min of stimulation. The retrieval efficiency value was normalised to a ratio of 1.0 for 30 mM KCl.

1.1.7 Glutamate Release Assay

The glutamate release assay was performed using enzyme-linked fluorescent detection of released glutamate (Cousin and Robinson., 2000a, b). Briefly, synaptosomes (0.6 mg in 2 ml) were resuspended in either plus (1.2 mM CaCl₂) or minus (1 mM EGTA) Ca²⁺ Krebs-like solution (118.5 mM NaCl, 4.7 mM KCl, 1.18 mM MgCl₂, 0.1 mM Na₂HPO4, 20 mM Hepes, 10 mM glucose, pH 7.4) at 37° C. Experiments were started after addition of 1 mM NADP⁺. After 1 minute 50 U of glutamate dehydrogenase was added and the synaptosome suspension was stimulated after 4 minutes with 30 mM KCl. Increases in fluorescence due to production of NADPH were monitored in a Perkin-Elmer LS-50B spectrofluorimeter at 340 nm excitation and 460 nm emission. Experiments were standardised by the addition of 4 nmol of glutamate. Data is presented as Ca²⁺-dependent glutamate release, calculated as the difference between release in plus and minus Ca²⁺ solution for identical stimulation conditions. In experiments using inhibitors, synaptosomes were preincubated for 5 min with either tyrphostin A47 or bis-tyrphostin before stimulation with KCl.

1.1.8 Electron Microscopy

Synaptosomes were incubated for 5 min in Krebs-like solution containing 1.2 mM Ca²⁺ then stimulated with 30 mM KCl for 2 minutes. Synaptosomes were preincubated with 100 μM bis-tyrphostin 5 minutes prior to KCl addition where indicated. After stimulation, synaptosomes were pelleted in a microfuge for 1 minute at room temperature then fixed by gentle resuspension in ice-cold phosphate buffered saline supplemented with 5% glutaraldehyde. After 1 hr they were centrifuged at low speed (2500 rpm) for 5 min at room temperature to loosely pellet the synaptosomes. The pellets were washed gently 3 times with MOPs buffer with low spins (2500 rpm) for 7 minutes then gently resuspended in a 10% bourine serum albumin (BSA) in water and allowed to stand for 20 min at room temperature. The synaptosomes were then centrifuged again for 7 minutes at low speed (2500 rpm), overlaid with Karnovsky's fixative and incubated at 4° C. overnight. The pellets were subsequently rinsed and fixed in a buffered solution of osmium tetroxide for 3 hours. Synaptosomes were then rinsed and stained for 1 hour in 2% aqueous uranyl acetate prior to being dried by a series of sequential 10 minute washes: 50% ethanol plus 0.1% NaCl, 70% ethanol plus 0.1% NaCl, 95% ethanol plus 0.1% NaCl, 100% ethanol plus 0.1% NaCl twice and 100% acetone twice. They were then infiltrated with an acetone/resin mixture (1:1) for 1 hour, washed 3 times for 10 minutes in Spur's epoxy resin at 70° C., then embedded within flat molds filled with Spur's epoxy resin for 10 hours at 70° C.

An ultramicrotome Ultracut-E (Reichert, Germany) was used to obtained 0.5 m epoxy sections from the resin blocks. The sections were cut with a diamond knife (Diatome, Switzerland), floated on water drops, placed on electron microscopy grids and double stained: first using 2% uranyl acetate in ethanol for 15 minutes and then Reynold's lead citrate for 4 minutes. The grids were washed in water, touch dried using absorbent filter paper and stored until analysis with an electron microscope. Analyses were performed on a Phillips 1L-BioTwin (Einhoven, Netherlands) electron microscope and pictures taken were printed on electron microscope plate film (Kodak, 4489, 8.3 cm×10.2 cm).

1.2 Results

1.2.1 Bis-tyrphostin Inhibits the GTPase Activity of Both Dynamin I and Dynamin II

The GTPase activity of dynamin plays an essential role in the ability of vesicles to bud from the plasma membrane during endocytosis. To initially find an inhibitor of the neuron-specific dynamin I a number of protein kinase inhibitors and some lipid kinase inhibitors which are highly potent ATPase active site-directed inhibitors were tested. These compounds were selected on the basis of the hypothesis that as ATPase active sites are similar to GTP active sites, then some ATPase inhibitors may also target dynamin. The results obtained showed some success with low potency inhibition of dynamin I GTPase activity.

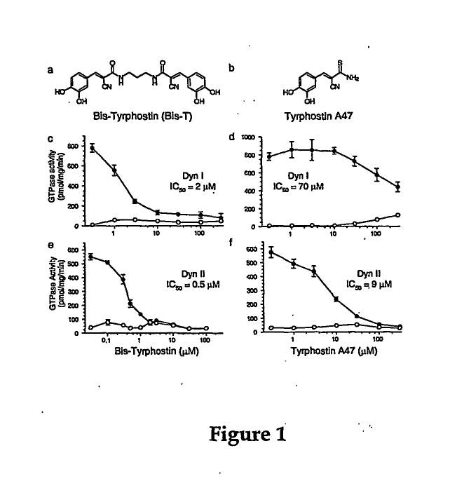

A series of tyrphostins were then evaluated and two were found that showed inhibition, namely tyrphostin A47 (IC₅₀=100 μM) and the most potent inhibitor of this sampling bis-tyrphostin, which showed an IC₅₀ of 2 μM (see FIG. 1c and 1d).

Typhostin A47 and bis-tyrphostin were subsequently tested for the purpose of evaluating whether the observed inhibition was specific to dynamin I, or if it also affected the ubiquitous dynamin II. Both drugs proved to be more potent for dynamin II (see FIG. 1e and 1f). More particularly, tyrphostin A47 showed an IC₅₀ of 9 M for dynamin II while bis-tyrphostin showed an IC₅₀ of just 0.5 μM. This indicates that a drug specific to each dynamin gene product may be designed thereby allowing for the pharmaceutical control of various forms of endocytosis.

1.2.2 Bis-tyrphostin and Tyrphostin A47 do not Prevent GTP Binding to Dynamin I or Dynamin II

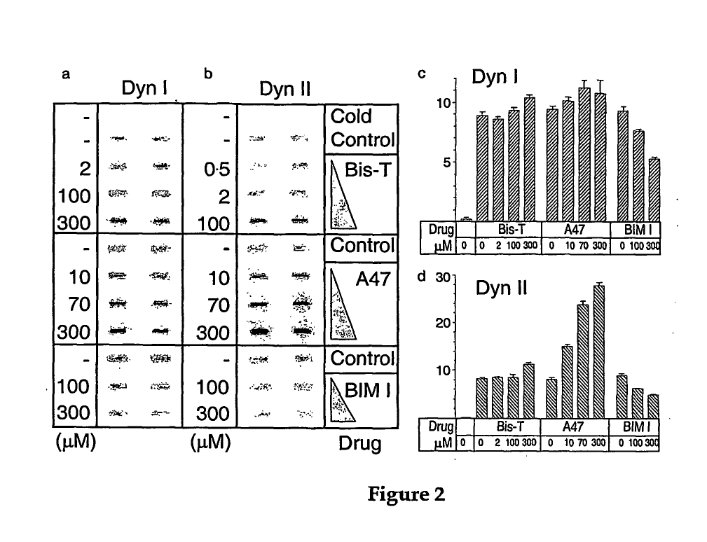

To evaluate the mechanism of action of bis-tyrphostin and tyrphostin A47 in preventing GTP hydrolysis by dynamin, the drugs were tested to see if they were competing with GTP at the active site on dynamin. [α-³²P]-GTP binding assays were completed to visualise radiolabelled GTP binding to dynamin in the presence or absence of bis-tyrphostin, tyrphostin A47 or BIM I (FIG. 2a-d). The controls (no drug) showed that [α-³²P]-GTP did bind to dynamin. In the presence of bis-tyrphostin and tyrphostin A47, GTP binding was not seen to decrease but, at high concentrations, was seen to actually be enhanced. This is especially so in the case of tyrphostin A47 vastly increasing GTP binding to dynamin II at high concentrations. The GTPase inhibitor BIM I was also found to compete with GTP for binding to dynamin as seen by the decrease in [α-³²P]-GTP binding.

Competition of these drugs with GTP for the active site of 4 small G proteins (Rab3A, Ras, Arf2, RalA) was also tested. It was found that neither drug affected [α-³²P]-GTP binding to these proteins (data not shown). This indicates that bis-tyrphostin and tyrphostin A47 are likely to be specific in their action to dynamin and not other G proteins which may be present in the nerve terminal or cell.

1.2.3 Bis-tyrphostin does not Act at the PH Domain of Dynamin I

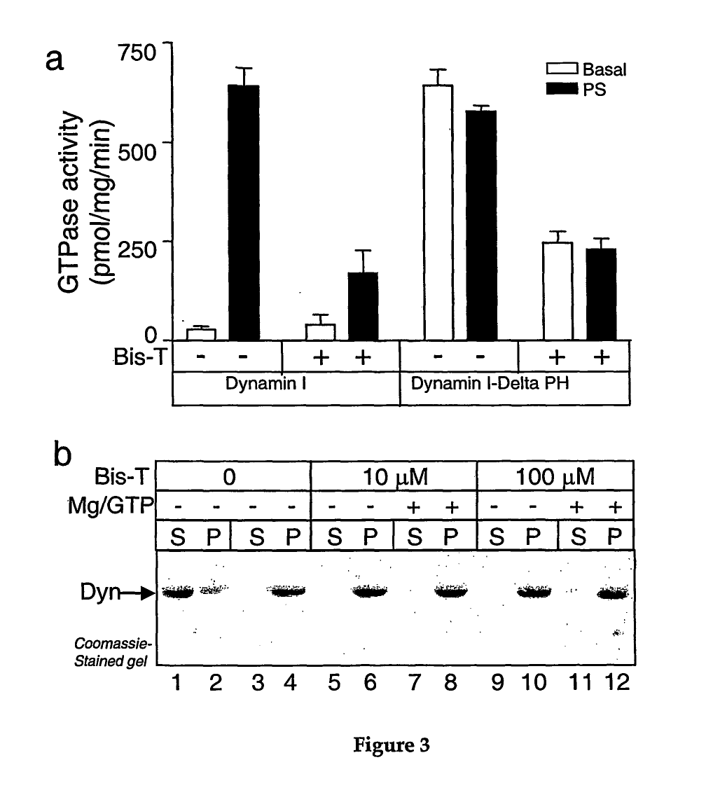

In order to determine if bis-tyrphostin was inhibiting dynamin I via its PH domain, the effect of bis-tyrphostin on a recombinant version of dynamin I lacking the PH domain (ΔPH domain dynamin I) was compared to its effect on wild type dynamin I (FIG. 3a). The PH domain of dynamin I acts as a negative regulator of its GTPase activity, ΔPH domain dynamin I is constitutively active and not affected by phospholipids. The results show that bis-tyrphostin was still able to inhibit ΔPH domain dynamin I GTP hydrolysis more than 50% at 10 μM. This shows that the PH domain is not the site of action of bis-tyrphostin on dynamin I which means that bis-tyrphostin must be inhibiting at an allosteric site on the dynamin I molecule. BIM I, however, lost its ability to inhibit dynamin I GTPase activity with the removal of the PH domain showing that this drug does prevent GTP hydrolysis via the PH domain.

Dynamin interaction with phospholipids stimulates GTPase activity by inducing cooperative dynamin helix assembly. Assembled dynamin is readily detected by a simple sedimentation assay and this characteristic was used to determine whether bis-tyrphostin regulates dynamin helix assembly or phospholipid interaction. Dynamin alone does not sediment in the assay and is retained in the supernatant (FIG. 3b, lanes 1-2), while it is found largely in the pellet in the presence of PS liposomes (lanes 3-4). Phospholipid binding, and hence dynamin helix assembly, was completely unaffected by 10 or 100 μM bis-tyrphostin (lane 5-12). Mg/GTP was added to the assay but did not alter the result. This indicates that bis-tyrphostin does not prevent dynamin association with phospholipids, nor its cooperative assembly. Hence, bis-tyrphostin inhibits dynamin GTPase activity at an allosteric site, and that it inhibits after the helix has assembled.

1.2.4 Bis-tyrphostin, but not Tyrphostin A47, Inhibits Dynamin I-mediated Synaptic Vesicle Retrieval, Forming Dynamin I Rings in the Process

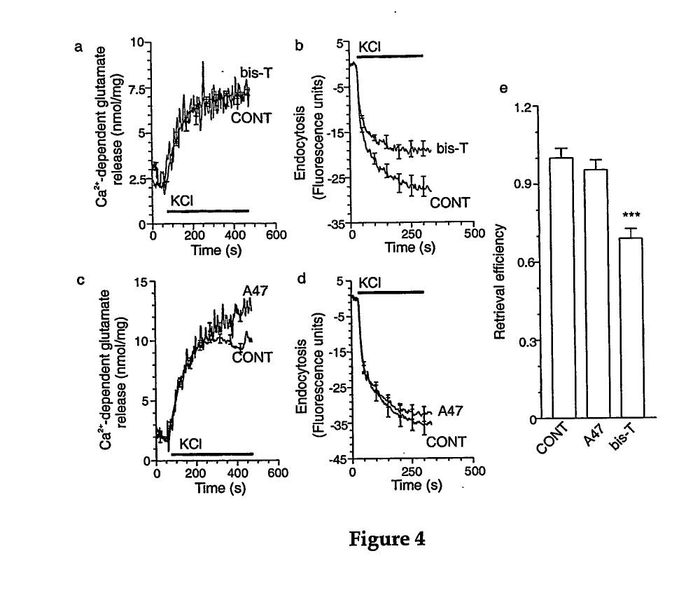

Fluorimetry was use to determine the effect of bis-tyrphostin and tyrphostin A47 on SVE in a population of rat brain nerve terminals (synaptosomes, FIG. 4). Bis-tyrphostin and A47 had no effect on exocytosis (Ca²⁺-dependent glutamate release, FIG. 4a and 4b). Bis-tyrphostin (100 μM for 10 min) significantly inhibited SVE, whereas A47 (100 μM) did not (FIG. 4c and d). Since the amount of SVE detected in this assay is dependent on the prior extent of exocytosis, the inhibition of endocytosis was quantified by calculating retrieval efficiency. This parameter is a ratio of the amount of endocytosis divided by the amount of exocytosis for each drug (Cousin et al., 2001). A retrieval efficiency of 1 indicates no drug effect on endocytosis. Tyrphostin A47 produced a retrieval efficiency of 0.95 (±0.05) and bis-tyrphostin of 0.7 (±0.05, FIG. 4e). This indicates a significant reduction in SVE by bis-tyrphostin.

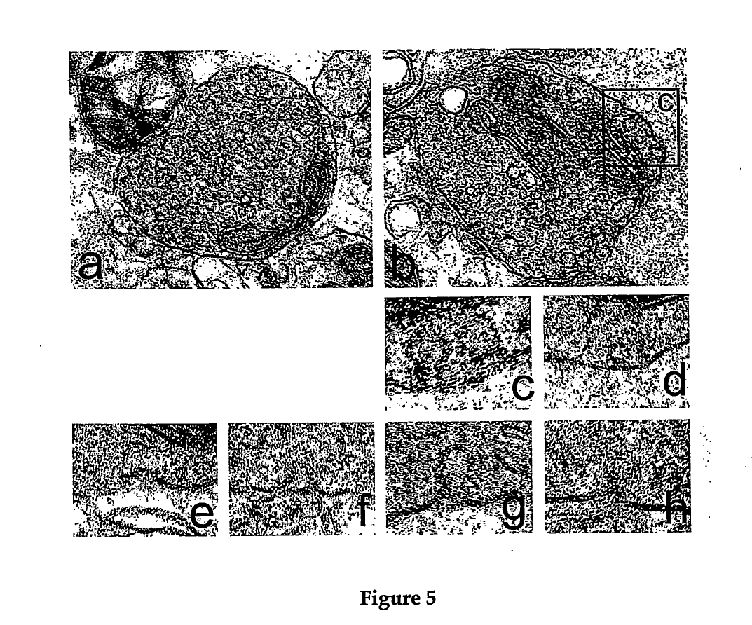

Since bis-tyrphostin inhibits dynamin I GTPase activity (FIG. 1), but not GTP binding (FIG. 2), a study was undertaken to determine whether bis-tyrphostin might also trap dynamin at the specific stage in SVE wherein it assembles as rings around the necks of budding synaptic vesicles. Synaptosomes at rest or depolarised once for 10 sec in 41 mM KCl (S1) exhibited normal morphology by electron microscopy (EM) (FIG. 5a-b). Nerve terminals were characterised by: i) a smooth, sealed plasma membrane, ii) they were completely filled with small synaptic vesicles, and iii) they almost always contained one to three normal mitochondrial profiles and occasionally contained a synapse and associated postsynaptic density. When unstimulated synaptosomes were treated with bis-tyrphostin there was no effect on their morphology (FIG. 5a). However, when depolarised there was a massive depletion of synaptic vesicles (FIG. 5b). A small number of plasma membrane invaginations were also detected (FIG. 5e-f), suggestive of failed endocytosis. As predicted for a blocker of GTP hydrolysis but not GTP binding, a number of collared pits were observed, with vesicle necks dearly encircled by dense collars (FIG. 5c, d, g and h).

1.2.5 Bis-tyrphostin Blocks the Dynamin II-mediated Receptor-mediated Endocytosis of Transferrin into Swiss 3T3 Cells and HER14 Cells

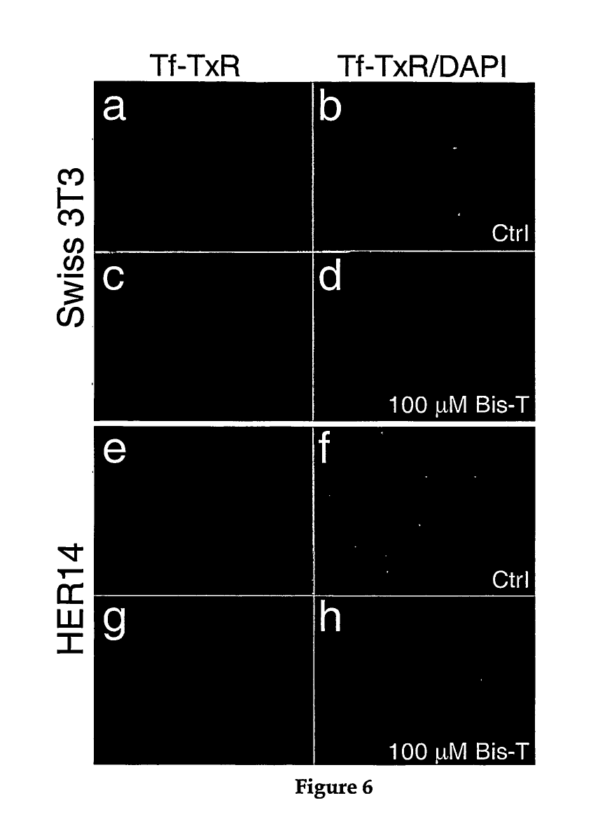

Transferrin is transported into cells by the process of receptor-mediated endocytosis which is mediated by dynamin II. The effect of both bis-tyrphostin and tyrphostin A47 on transferrin internalisation into non-neuronal cells was tested (FIG. 6). Control cells showed a large degree of cytoplasmic staining (panels a and e) indicating that transferrin has been internalised into the cells. The cell nuclei were co-stained in blue with DAPI to indicate the location of the cell bodies (panels b, d, f and h). Upon addition of bis-tyrphostin a very large decrease in transferrin staining was observed. Tyrphostin A47 also produced this effect though not as dramatically as bis-tyrphostin (not shown). The inhibition was also found to be concentration-dependent. The vehicle DMSO had no effect on transferrin internalisation.

1.3 Discussion

As first demonstrated in the mutant Drosophila strain shibire, blocking dynamin and endocytosis in nerve terminals results in a dramatic depletion of most SVs. Since the large number of SVs are one of the most defining morphological features of nerve terminals their loss is readily evident visually. Furthermore, the resulting morphology of the plasma membrane is known to provide a strong indication of the point in endocytosis at which the block is occurring. Bis-tyrphostin depleted nerve terminals of most SVs and produced a very small number of vesicles trapped on the plasma membrane with clear dynamin collars or rings around their necks. This dramatic result revealed that the site of action of bis-tyrphostin follows ring assembly and before neck fission. However, surprisingly, dynamin collars were rare. This surprising complexity suggests bis-tyrphostin blocks at a second point prior to ring assembly providing support that dynamin GTPase activity is important at two distinct points in the mechanisms of SVE.

The three dynamin gene products may mediate at least 3 forms of endocytosis. Dynamin I mediates SVE, dynamin II mediates RME and dynamin m may mediate endocytosis in postsynaptic spines (Gray et al., 2003). Further mechanistic subtleties are also known. Differential inhibition of the dynamins provides the capability of distinguishing between these cellular roles. In particular, a selective inhibitor is an important tool for discriminating between different types of endocytosis and has clinical interest for targeting pathology based on the different forms of endocytosis.

The results further indicate that bis-tyrphostin (BisT or AG537) inhibits the GTPase activity of dynamins I and II and blocks both SVE in nerve terminals (synaptosomes) and RME of transferrin in 3T3 or HER14 cells. Its site of action is not the GTP binding site nor the PH domain and so it is an allosteric inhibitor. Since it does not affect GTP binding it also should not affect dynamin assembly into rings. This provides a unique tool that targets dynamin after it has assembled. Bis-tyrphostin has previously been found to inhibit EGFR-TK (IC₅₀=0.4 M) and EGF-dependent cell proliferation (IC₅₀=3 M) (Gazit et al., 1996). Therefore, analogues were designed that retained dynamin inhibition, but which lose their effect on EGFR-tyrosine kinase (since the determinants for tyrosine kinase specificity are well known (Gazit et al., 1996).

### EXAMPLE 2

### Development of Tyrophostin Analogues

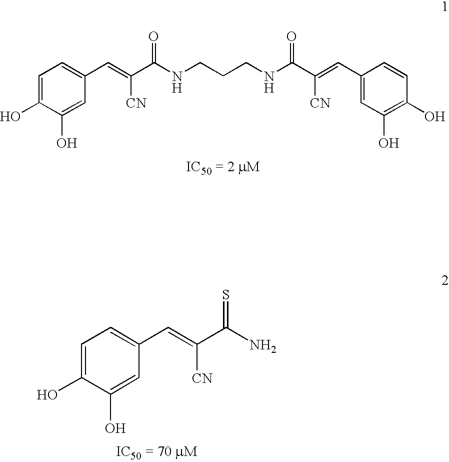

Structures of bis-tyrphostin (1) and tyrphostin A47 (2).

2.1 Development of Analogues

The structure for bis-tyrphostin and tyrphostin A47 are shown above. The structural similarities between these compounds of the 3,4-dihydroxybenzene and the presence of the cyanoamide or thioamide suggested that these groups may be important for dynamin inhibition. These features are highly amenable to solution phase parallel synthesis approaches to library development and two libraries were synthesised to determine type and number of aromatic substituents crucial for activity, the requirement for symmetrical systems (1 vs 2), and the importance of the length of the central alkane spacer arm between the two amide moieties present in bis-tyrphostin. These libraries were termed library 1 (dimeric compounds) and library 2 (asymmetric, monomeric compounds).

2.2 Synthesis of Analogue Libraries

Simple application of Knoevenagel chemistry and a series of appropriate α,w-bisamines rapidly afforded the desired libraries (Scheme 1) in good to excellent yields.

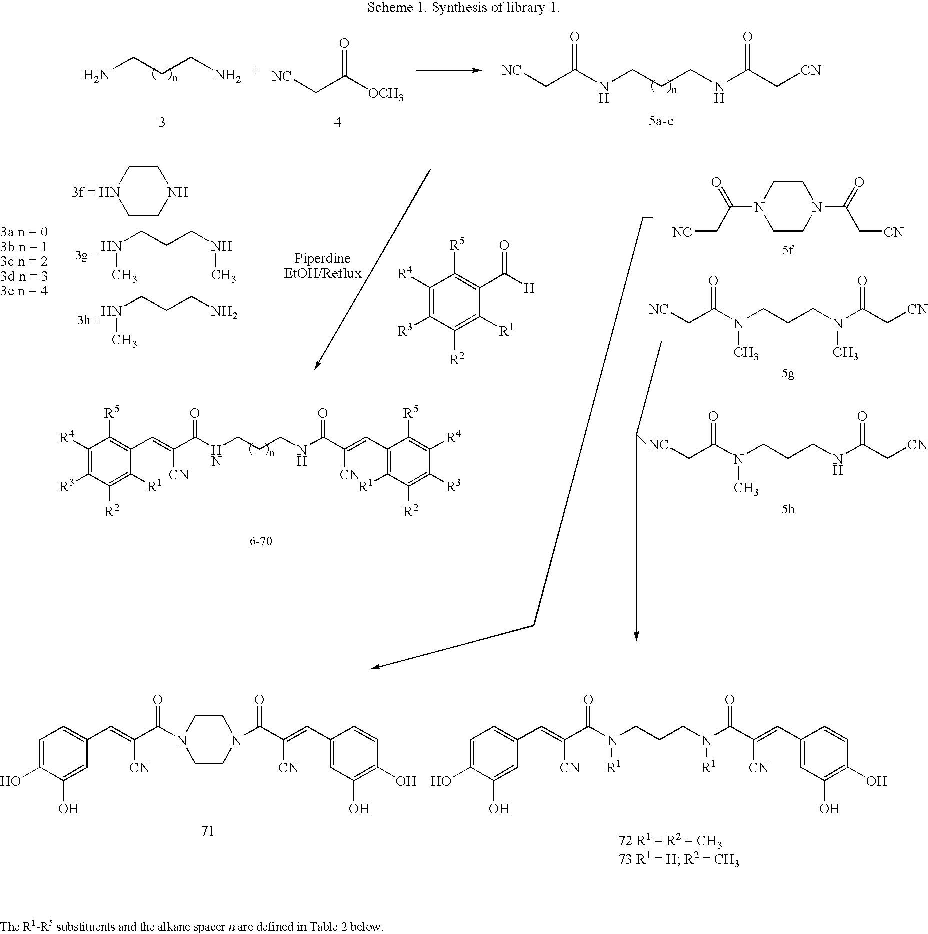

Utilisation of this approach allowed the rapid generation of five discreet sub-libraries within library 1, based upon the length of the alkane spacer arm with n=1-5. Initial biological screens for dynamin I GTPase activity were conducted at 100 μM. More promising analogues were then screened across a range of concentrations to determine their IC₅₀ values (Table 2). Of the 80 analogues synthesized a number of compounds were found to have an IC₅₀ of 100 μM or below and exhibited marked inhibition. The R₁ to R₅ substituents are identified in Table 1 below.

2.3 Synthesis of Dimeric Tyrphostins

2.3.1 General

All starting materials were purchased from Aldrich Chemical Company and Lancaster Synthesis. ¹H and ¹³C spectra were recorded on a Bruker Advance AMX 300 MHz spectrometer at 300.1315 and 75.4762 MHz respectively. Chemical shifts are relative to TMS as internal standard.

2.3.2 Synthetic Methods

Compound 5a: 2-Cyano-N-[3-(2-cyanoacetylamino)-ethyl]-acetamide

Ethylenediamine (3a) (1.5 g, 25 mmol) and methylcyanoacetate (5 g, 50 mmol) were stirred at room temperature for 2 hours. The resulting white solid was then mixed with 10 mL ethanol and collected by filtration. Recrystallization from ethanol gave a white solid, 6.3 g (81%). mp 182° C. (Lit 183° C.)²⁹.

¹H NMR (DMSO): 8.25 (2H, t, J=5.5 Hz), 3.56 (4H, s), 3.13 (4H, br s). ¹³C NMR (DMSO): 162.31, 115.96, 38.41, 25.25.

Compound 5b: 2-Cyano-N-[3-(2-cyanoacetylamino)-propyl]-acetamide

Propanediamine (3b) (2.2 g, 30 mmol) and methylcyanoacetate (6.4 g, 65 mmol) were stirred at room temp for two hours The resulting white solid was then mixed with 20 mL of ethanol and collected by filtration. Recrystallization from ethanol gave 4.995 g of white solid (81%). mp 146° C. (Lit 148° C.)²⁹

¹H NMR (DMSO): 8.21 (2H, t, J=5.5 Hz), 3.59 (4H, s), 3.07 (4H, q, J=6.7 Hz), 1.53 (2H, quin, J=6.7 Hz). ¹³C NMR (DMSO): 162.45, 116.64, 39.28, 28.90, 25.67.

Compound 5c: 2-Cyano-N-[3-(2-cyanoacetylamino)-butyl]-acetamide

1,4-diaminobutane (3c) (3 g, 34 mmol) and methylcyanoacetate (7 g, 70 mmol) were stirred at room temp for two hours after which time a white solid was formed. The solid was then mixed with ethanol (10 mL) and collected by filtration. Recrysalization from ethanol gave a white solid, 5.995 g (78%). mp 145° C. (Lit 145° C.)

¹H NMR (DMSO): 8.15 (2H, t, J=5.5 Hz), 3.56 (4H, s), 3.05 (4H, br s), 1.38 (4H, br s) ¹³C NMR (DMSO): 161.84, 116.09, 38.63, 26.07, 25.17.

Compound 5d: 2-Cyano-N-[3-(2-cyanoacetylamino)pentyl]-acetamide

1,5-diaminopentane (3d) (2 g, 20 mmol) and methylcyanoacetate (3.9 g, 40 mmol) were stirred at room temp for two hours after which time a white solid was formed. The solid was then mixed with ethanol (10 mL) and collected by filtration. Recrysalization from ethanol gave a white solid, 4.62 g (98%). mp 125° C. (Lit 125° C.)

¹H NMR (DMSO): 8.14 (2H, t, J=5.4 Hz), 3.55 (s, 4H), 3.03 (4H, q, J=6.4 Hz), 1.39 (4H, quin, J=7 Hz), 1.23 (2H, quin, J=7 Hz). ¹³C NMR (DMSO): 161.79, 116.11, 38.84, 28.26, 25.17,23.43.

Compound 5e: 2-Cyano-N-[3-(2-cyanoacetylamino)-hexyl]-acetamide

1,6 diaminohexane (3e) (3 g, 26 mmol) and methylcyanoacetate (6 g, 60 mmol) were stirred at room temp for 2 hours after which time a white solid was formed. The solid was then mixed with ethanol (10 mL) and collected by filtration. Recrystalization from ethanol gave a white solid, 6.2 g (95%). mp 141° C. (Lit 140° C.)

¹H NMR (DMSO): 8.15 (2H, t, J=5.5 Hz), 3.56 (4H, s), 3.04 (4H, q, J=6.1 Hz), 1.37 (4H, quin, J=5.9 Hz), 1.24 (4H, br s). ¹³C NMR (DMSO): 161.76, 116.12, 38.85, 28.58, 25.82, 25.16.

Compound 9: 2-Cyano-N-{3-[2-cyano-3-(3,4-dihydroxyphenyl)-acryloylamino]-ethyl}-3-(3,4-dihydroxyphenyl)-acrylamide

2-Cyano-N-[3-(2-cyano-acetylamino)-ethyl]-acetamide (5a) (0.3 g, 1.5 mmol), 3,4-dihydroxybenzaldehyde (0.42 g, 3 mmol), 3 drops of piperidine and ethanol (10 mL) were refluxed for 2 hours. Cooling, filtering and washing with cold ether (10 mL) gave a yellow-green solid, 0.54 g (81%). mp 290° C. (Lit 295° C.)

¹H NMR (DMSO): 8.32 (2H, t, J=5.5 Hz), 7.92 (2H, s), 7.53 (2H, d, J=2.1 Hz), 7.25 (2H, dd, J=8.2, 2.1 Hz), 6.85 (2H, d, J=2.1 Hz), 3.45 (4H, br s). ¹³C NMR (DMSO): 162.50, 151.63, 161.61, 146.22, 125.76, 123.45, 117.65, 116.53, 116.31, 100.85, 39.60.

Compound 10: 2-Cyano-N-{3-[2-cyano-3-(3,4,5-trihydroxyphenyl)-acryloylamino]-ethyl}-3-(3,4,5-trihydroxyphenyl)-acrylamide

2-Cyano-N-[3-(2-cyano-acetylamino)-ethyl]-acetamide (5a) (0.056 g, 0.3 mmol), 3,4,5-trihydroxybenzaldehyde (0.1 g, 0.65 mmol) and 1 drop piperidine and ethanol (2 mL) were refluxed for 1 hour. Cooling, filtering and washing with cold ethanol (10 mL) gave an orange solid, 0.11 g (82%). mp>300° C.

¹H NMR (DMSO): 8.29 (2H, t, J=5.5 Hz), 7.79 (2H, s), 6.99 (4H, s), 3.32 (4H, br s). ¹³C NMR (DMSO): 162.15, 150.7, 145.96, 140.24, 121.26, 117.30, 109.97, 99.76, 39.40.

Compound 11: 2-Cyano-N-{3-[2-cyano-3-(3,4-dihydroxy-4-methoxyphenyl)-acryloylamino]-ethyl}-3-(3,4-dihydroxy-5-methoxyphenyl)-acrylamide

2-Cyano-N-[3-(2-cyano-acetylamino)-ethyl]-acetamide (5a) (0.06 g, 3 mmol), 3,4-dihydroxy-5-methoxybenzaldehyde (0.1 g, 0.6 mmol), 1 drop of piperidine and 2 mL of ethanol were refluxed for 2 hours. Cooling, filtering and washing with cold ethanol (5 mL) gave an orange solid, 0.101 g (66%). mp 274° C.

¹H NMR (DMSO): 8.34 (2H, t, J=5.5 Hz), 7.93 (1H, s), 7.20 (2H, d, J=1.92 Hz), 7.13 (2H, d, J=1.92 Hz), 3.77 (6H, s), 3.35 (4H, br s). ¹³C NMR (DMSO): 161.90, 150.85, 148.03, 145.83, 139.90, 121.76, 117.20, 111.09, 107.20, 100.83.

Compound 22: 2-Cyano-N-{3-[2-cyano-3-(3,4-dihydroxyphenyl)-acryloylamino]-propyl}-3-(3,4-dihydroxyphenyl)-acrylamide

2-Cyano-N-[3-(2-cyanoacetylamino)propyl]-acetamide (5b) (0.3 g 1.4 mmol), (0.4 g, 2.8 mmol) 3,4-dihydroxybenzaldehyde, 3 drops of piperidine and 10 mL of ethanol were refluxed for 2 hours. Cooling, filtering and washing with cold ether (10 mL) gave a yellow green solid, 0.55 g (85%). mp 274° C. (Lit 277° C.)

¹H NMR (DMSO): 8.24 (2H, t, J=5.5 Hz), 7.92 (s, 2H), 7.52 (2H, d, J=2.1 Hz), 7.26 (2H, dd, J=8.2, 2.1 Hz), 6.85 (2H, d, J=8.2 Hz), 3.23 (4H, q, J=6 Hz), 1.70 (2H, quin, J=6.7 Hz). ¹³C NMR (DMSO): 161.50, 150.60, 150.50, 125.10, 123.21, 117.10, 116.00, 115.80, 100.50, 37.27, 28.82.

Compound 23: 2-Cyano-N-{3-[2-cyano-3,4,5-trihydroxyphenyl)-acryloylamino]-propyl}-3-(3,4,5-trihydroxyphenyl)-acrylamide

2-Cyano-N-[3-(2-cyanoacetylamino)propyl]-acetamide (5b) (0.06 g 0.29 mmol), 3,4,5-trihydroxybenzaldehyde (0.1 g, 0.58 mmol), 1 drop of piperidine and ethanol (10 mL) were refluxed for 2 hours. Cooling, filtering and washing with cold ethanol (10 mL) gave an orange solid, 0.097 g (70%). Mp>300° C. (Lit>300° C.)

¹H NMR (DMSO): 8.18 (2H, t, J=5.5 Hz), 7.78 (2H, s), 6.99 (4H, s), 3.21 (4H, q, J=6.8 Hz), 1.68 (2H, quin, J=6.8 Hz). ¹³C NMR (DMSO): 161.80, 150.70, 145.95, 140.30, 121.22, 117.30, 109.90, 99.50, 38.20, 28.90.

Compound 24: 2-Cyano-N-{3-[2-cyano-3-(3,4-dihydroxy-methoxyphenyl)-acryloylamino]-propyl}-3-(3,4-dihydroxy-5-methoxyphenyl)-acrylamide

2-Cyano-N-[3-(2-cyanoacetylamino)-propyl]-acetamide (5b) (0.3 g 1.4 mmol), 0.44 g 3,4-dihydroxy-4-methoxybenzaldehyde, 3 drops of piperidine and ethanol (10 mL) were refluxed for 2 hours. Cooling, filtering and washing with cold ethanol (5 mL) gave an orange solid, 0.31 g (42%). mp>300° C.

¹H NMR (DMSO): 8.35 (2H, t, J=5.4 Hz), 7.95 (2H, s), 7.21 (2H, d, J=1.9 Hz), 7.12 (2H, d, J=1.9 Hz), 3.21 (4H, q, J=6.8 Hz), 1.71 (2H, quin, J=6.8 Hz). ¹³C NMR (DMSO): 161.30, 150.61, 147.20, 145.30, 121.04, 117.60, 110.60, 107.65, 98.71, 38.35, 28.88.

Compound 35: 2-Cyano-N-{3-[2-cyano-3-(3,4-dihydroxyphenyl)-acryloylamino]-butyl}-3-(3,4-dihydroxyphenyl)-acrylamide

2-Cyano-N-[3-(2-cyanoacetylamino)-butyl]-acetamide (5c) (0.3 g, 1.35 mmol), 3,4-dihydroxybenzaldehyde (0.37 g, 2.7 mmol), 3 drops of piperidine and ethanol (10 mL) were refluxed for 2 hours. Cooling, filtering and washing with cold ether (10 mL) gave a yellow solid, 0.61 g (97%). mp 281° C. (Lit 283° C.)

¹H NMR (DMSO): 8.25 (2H, t, J=5.5 Hz), 7.91 (2H, s), 7.53 (2H, d, J=1.9 Hz), 7.26 (2H, dd, J=8.3, 1.9 Hz), 6.85 (2H, d, J =8.3 Hz), 3.20 (4H, br s), 1.49 (4H, br s). ¹³C NMR (DMSO): 161.52, 150.86, 150.42, 145.65, 125.23, 123.09, 117.20, 115.81, 100.51, 39.31.

Compound 36: 2-Cyano-N-{3-[2-cyano-3-(3,4,5-trihydroxyphenyl)acryloylamino]-butyl}-3-(3,4,5-trihydroxyphenyl)-acrylamide

2-Cyano-N-[3-(2-cyanoacetylamino)-butyl]-acetamide (5c) (0.065 g, 0.3 mmol), 3,4,5-trihydroxybenzaldehyde (0.1 g, 0.6 mmol), 1 drop of piperidine and ethanol (2 mL) were refluxed for 1 hour. Cooling, filtering and washing w2-Cyano-N-{3-[2-cyano-3-(3,4,5-trihydroxyphenyl)-acryloylamino]-butyl}-3-(3,4,5-trihydroxyphenyl)-acrylamide ith cold ether (5 mL) gave a yellow solid, 0.121 g (82%). mp>300° C. (Lit>310° C.)²⁹

¹H NMR (DMSO): 8.16 (2H, t, J=5.5 Hz), 7.78 (2H, s), 6.98 (4H, s), 3.19 (4H, br s), 1.48 (4H, br s). ¹³C NMR (DMSO): 161.70, 150.56, 145.90, 140.20, 121.30, 117.30, 109.90, 99.80, 39.26, 26.37.

Compound 37: 2-Cyano-N-{3-[2-cyano-3-(3,4-dihydroxy-5-methoxyphenyl)-acryloylamino]-butyl}-3-(3,4-dihydroxy-5-methoxyphenyl)acrylamide

2-Cyano-N-[3-(2-cyanoacetylamino)butyl]-acetamide (5c) (0.065 g, 0.3 mmol), 3,4-dihydroxy-5-methoxybenzaldehyde (0.1 g, 0.6 mmol), 1 drop of piperidine and ethanol (2 mL) were refluxed for 1 hour. Cooling, filtering and washing with cold ether (5 mL) gave a yellow solid, 0.110 g (70%). mp>300° C.

¹H NMR (DMSO): 8.09 (2H, t, J=5.5 Hz), 7.86 (2H, s), 7.18 (21H, d, J=1.9 Hz), 7.10 (2H, d, J=1.9 Hz), 3.75 (6H, s), 3.19 (4H, br s), 1.48 (4H, br s). ¹³C NMR (DMSO): 161.71, 150.23, 148.70, 146.24, 120.25, 117.51, 109.50, 106.80, 98.81, 55.76, 39.31, 26.64.

Compound 48: 2-Cyano-N-{3-[2-cyano-3-(3,4-dihydroxyphenyl)-acryloylamino]-pentyl}-3-(3,4-dihydroxyphenyl)-acrylamide

2-Cyano-N-[3-(2-cyanoacetylamino)-pentyl]-acetamide (5d) (0.2 g, 0.85 mmol), 3,4-dihydroxybenzaldehyde (0.23 g, 1.7 mmol), 3 drops of piperidine and 7 mL ethanol were refluxed for 2 hours. Cooling, filtering and washing with cold ether (10 mL) gave a yellow solid, 0.36 g (90%). mp 252° C. (Lit 248° C.)²⁹

¹H NMR (DMSO): 8.15 (2H, t, J=5.5 Hz), 7.85 (2H, s), 7.50 (2H, d, J=2.1 Hz), 7.20 (2H, dd, J=8.5 Hz, 2 Hz), 6.75 (2H, d, J=8.5 Hz), 3.16 (4H, q, J=6.2 Hz), 1.50 (4H, quin, J=7.1 Hz), 1.28 (2H, quin, J=6.9 Hz). ¹³C NMR (DMSO): 161.85, 153.88, 150.34, 146.28, 126.16, 121.47, 117.70, 115.71, 114.65, 98.40, 39.46, 28.63, 23.73.

Compound 49: 2-Cyano-{3-[2-cyano-3-(3,4,5-trihydroxyphenyl)-acryloylamino]-pentyl}-3-(3,4,5-trihydroxyphenyl)-acrylamide

2-Cyano-N-[3-(2-cyanoacetylamino)-pentyl]-acetamide (5d) (0.068 g, 0.29 mmol), (0.1 g, 058 mmol) 3,4,5-trihydroxybenzaldehyde (0.1 g, 0.58 mmol), 1 drop of piperidine and ethanol (2 mL) were refluxed for 1 hour. Cooling, filtering and washing with cold ether (5 mL) gave a yellow solid, 0.123 g (83%). mp>300° C.³²

¹H NMR (DMSO): 8.12 (2H, t, J=5.5 Hz), 7.76 (2H, s), 6.98 (4H, s), 3.16 (4H, br s), 1.50 (4H, quin, J=6.8 Hz), 1.28 (2H, quin, J=6.7 Hz). ¹³C NMR (DMSO): 161.80, 150.49, 146.11, 146.01, 141.25, 120.69, 117.53, 109.90, 99.12, 39.47, 28.62, 22.30.

Compound 50: 2-Cyano-N-{3-[2-cyano-3-(3,4-dihydroxy-5-methoxyphenyl)-acryloylamino]-pentyl}-3-(3,4-dihydroxy-5-methoxyphenyl)-acrylamide

2-Cyano-N-[3-(2-cyanoacetylamino)-pentyl]-acetamide (5d) (0.069 g, 0.29 mmol) 3,4-dihydroxy-5-methoxybenzaldehyde (0.1 g, 0.58 mmol), 1 drop of piperidine and ethanol (2 mL) were refluxed for 1 hour. Cooling, filtering and washing with cold ether (5 mL) gave a yellow solid, 0.126 g (81%). mp 256° C.

¹H NMR (DMSO): 8.09 (2H, t, J=5.5 Hz), 7.86 (2H, s), 7.18 (2H, d, J=2 Hz), 7.10 (2H, d, J=2 Hz) 3.75 (6H, s), 3.17 (4H, br s), 1.50 (4H, quin, J=6.8 Hz), 1.28 (4H, quin, J=6.9 Hz). ¹³C NMR (DMSO): 161.81, 150.50, 148.00, 146.20, 120.05, 117.81, 110.50, 107.80, 98.71, 55.71, 39.41, 28.64, 22.47.

Compound 61: 2-Cyano-N-{3-[2-cyano-3-(3,4-dihydroxyphenyl)-acryloylamino]-hexyl}-3-(3,4-dihydroxyphenyl)-acrylamide

2-Cyano-N-[3-(2-cyanoacetylamino)hexyl]-acetamide (5e) (0.3 g, 1.2 mmol), 3,4-dihydroxybenzaldehyde (0.33 g, 2.4 mmol), 3 drops of piperidine and 10 mL ethanol were refluxed for 2 hours. Cooling, filtering and washing with cold ether (10 mL) gave a yellow solid, 0.52 g (89%). mp 263° C. (Lit 260° C.)

¹H NMR (DMSO): 8.18 (2H, t, J=5.5 Hz), 7.89 (2H, s), 7.51 (2H, d, J=2 Hz), 7.24 (2H, dd, J=2 Hz, 8.3 Hz), 6.83 (2H, d, J=8.3 Hz), 3.17 (4H, q, J=6.1 Hz), 1.47 (4H, quin, J=6.1 Hz), 1.28 (4H, br s). ¹³C NMR (DMSO): 161.52, 151.18, 150.30, 145.72, 125.22, 122.91, 117.24, 115.80, 115.72, 100.38, 39.48, 28.78, 25.98.

Compound 62: 2-Cyano-N-{3-[2-cyano-3-(3,4,5-trihydroxyphenyl)-acryloylamino]-hexyl}-3-(3,4,5-trihydroxyphenyl)-acrylamide

2-Cyano-N-[3-(2-cyanoacetylamino)hexyl]-acetamide (5e) (0.073 g, 0.29 mmol), 3,4,5-trihydroxybenzaldehyde (0.1 g, 0.58 mmol), 1 drop of piperidine and ethanol (2 mL) were refluxed for 1 hour. Cooling, filtering and washing with cold ether (5 mL) gave a yellow solid, 0.1 g (67%). mp>300° C.

¹H NMR (DMSO): 8.11 (2H, t, J=5.5 Hz), 7.76 (2H, s), 6.98 (4H, s), 3.16 (4H, br s). 1.47 (4H, quin, J=6.1 Hz), 1.28 (4H, br s). ¹³C NMR (DMSO): 161.80, 150.46, 145.99, 141.19, 120.71, 117.53, 109.89, 99.15, 39.68, 28.84, 26.01.

Compound 63: 2-Cyano-N-{3-[2-cyano-3-(3,4-dihydroxy-5-methoxyphenyl)-acryloylamino]-hexyl}-3-(3,4-dihydroxy-5-methoxyphenyl)-acrylamide

2-Cyano-N-[3-(2-cyanoacetylamino)hexyl]-acetamide (5e) (0.069 g, 0.28 mmol), 3,4,dihydroxy-5-methoxybenzaldehyde (0.1 g, 0.56 mmol), 1 drop of piperidine and ethanol (2 mL) were refluxed for 1 hour. Cooling, filtering and washing with cold ether (5 mL) gave a yellow solid, 0.132 g (86%). mp 243° C.

¹H NMR (DMSO): 8.17 (2H, t, J=5.5 Hz), 7.89 (2H, s), 7.19 (2H, d, J=1.6 Hz), 7.13 (2H, d, J=1.6 Hz), 3.77 (6H, s), 3.17 (4H, br s), 1.48 (4H, quin, J=6.1 Hz), 1.29 (4H, br s). ¹³C NMR (DMSO): 161.80, 150.49, 146.11, 146.01, 141.25, 120.69, 117.53, 109.90, 99.12, 39.47, 28.62, 22.30.

2.3.2 Activity of Dimeric Tyrphostins

TABLE 1
Effect of library 1 (dimeric) compounds on dynamin I GTPase activity.

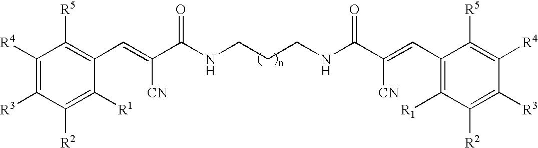

| | | | | | | | |
|---|---|---|---|---|---|---|---|
| Compound | R¹ | R² | R³ | R⁴ | R⁵ | n | IC₅₀ (μM)^{a} |

| | | | | | | | |
|---|---|---|---|---|---|---|---|
| 9 | H | H | OH | OH | H | 1 | 5.1 ± 0.6 |
| 10 | H | OH | OH | OH | H | 1 | 1.7 ± 0.2 |
| 11 | H | OMe | OH | OH | H | 1 | 9 ± 3 |
| 22 | H | H | OH | OH | | 2 | 1.7 ± 0.5 |
| 23 | H | OH | OH | OH | | 2 | 1.7 ± 0.2 |
| 24 | H | OMe | OH | OH | | 2 | 5 ± 1 |
| 35 | H | H | OH | OH | | 3 | 3.2 ± 1 |
| 36 | H | OH | OH | OH | | 3 | 2.1 ± 0.2 |
| 37 | H | OMe | OH | OH | | 3 | 8 ± 0.15 |
| 48 | H | H | OH | OH | | 4 | 5 ± 1.4 |
| 49 | H | OH | OH | OH | | 4 | 1.7 ± 0.4 |
| 50 | H | OMe | OH | OH | | 4 | 8 ± 0.15 |
| 61 | H | H | OH | OH | | 5 | 26 ± 15 |
| 62 | H | OH | OH | OH | | 5 | 6 ± 2 |
| 63 | H | OMe | OH | OH | | 5 | 80 ± 4 |

Mono-substituted aromatic compounds containing no substitutions, or single substituent such as a single —OH (eg, R₁ or R₂ is OH), —Cl (R₂ or R₄ is Cl), —OMe (R₂ or R₃ is OMe), or —COOH (R₃ is COOH) showed no dynamin inhibition. Introduction of a second oxygen-bearing substituent had a pronounced effect. The 3,4-di-OH (11, IC₅₀=5.1±0.6 μM) displayed similar potency to compound 1, namely bis-tyrphostin (2,3-di-OH). The 3,4,5-tri-substituted aromatic compound (10) also had equivalent potency to 1. Essentially the same trend was observed for each series of different chain length compounds.

Alkane spacer chain elongation had little effect on potency until n>3. For example, chain extended analogues of 9 (n=0), i.e. 22 (n=1), 35 (n=2), 48 (n=3), and 61 (n=4) displayed IC₅₀ values of 5.1±0.6, 1.7±0.2, 3.2±1, 5±1.4 and 26±1.5 μM, respectively. Essentially the opposite trend was previously reported for tyrosine kinase inhibition. Whilst examining compounds against EGF receptor tyrosine kinase phosphorylation of a poly-GAT substrate, Gazit et al observed that inhibition was independent of chain length (Gazit et al., 1996).

Analogues in which R₁ and the position occupied by the cyanyl group (CN) are cylised may also be provided. For instance, when R₁ is hydroxy, the hydroxy group can react with cyanyl to form an imminochromene as show in Scheme 2 below.

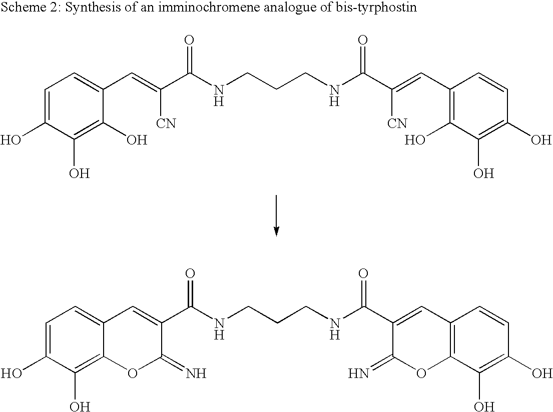

To attempt to explain similarities in inhibitory values for the chain-extended analogues of 9, modelling analysis of all 5 alkane spacer analogues was conducted and the resulting MacSpartanPro low energy conformer models are shown below. As can be seen, the low energy conformers of all 5 analogues adopt comparable hairpin conformations, maximizing pi-pi interactions between the terminal phenyl rings. Consequently, increasing the spacer length has limited impact until entrophic effects begin to impinge on the relative stability of the hairpin conformation (n≧5). This contrasts with the effects of dimeric tyrphostins on tyrosine kinase potency which resides in their extended configuration and thus allows them to fit the dimeric intermediate of the EGFR tyrosine kinases.

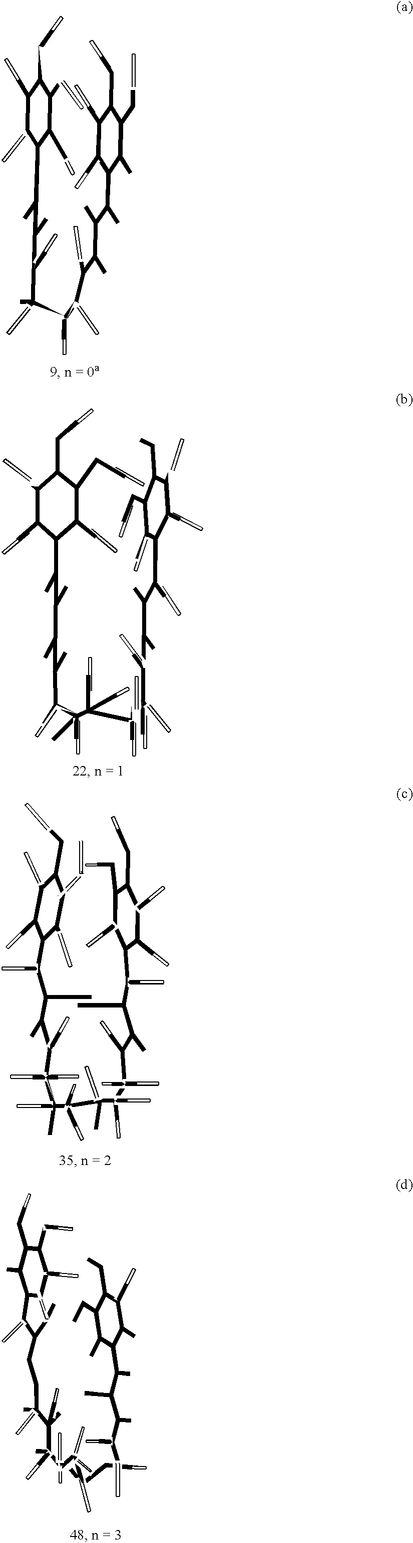

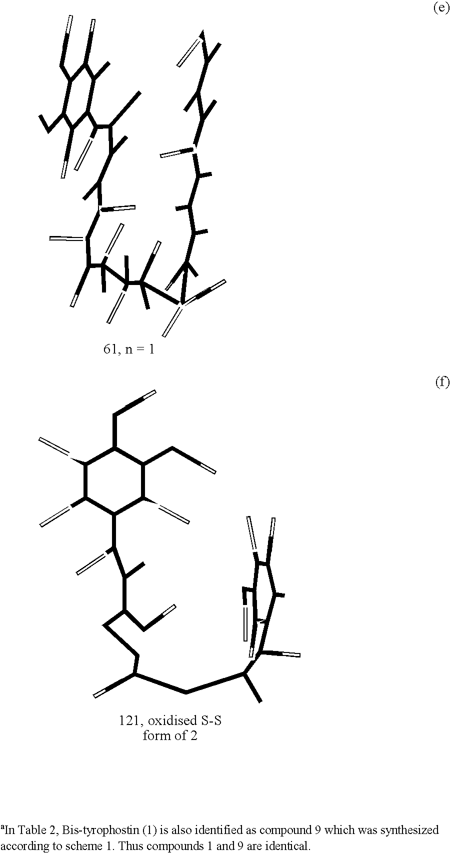

To explore the potential H-bonding effects associated with 1, compound 71 was developed. This compound has a relatively inflexible piperazine linker of similar overall size to 1. However, it displayed no dynamin inhibition at ≦100 μM. Similarly, no inhibitory effect was observed after N-methylating the alkane spacer of 1 to produce N-methyl analogue 72. These observations suggest that the hairpin conformation of dimeric tryphostins is desirable for inhibitory action supporting the modelling observations (hairpin conformation rather than extended chain), and that the amide substituents also play an important role in binding to dynamin.

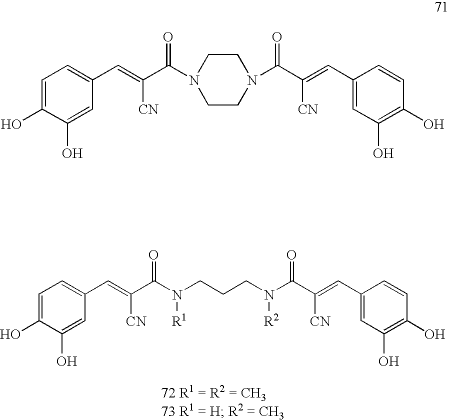

### Structures of Compounds 71-73

Having successfully developed a number of μM potent symmetrical analogues based on bis-tyrphostin, modifications of one of the aromatic nuclei were investigated to determine its role in inhibiting dynamin. Accordingly, another compound library based on tyrphostin A47 (2) was developed as shown in scheme 3, and the analogues ability to inhibit dynamin I GTPase activity was examined.

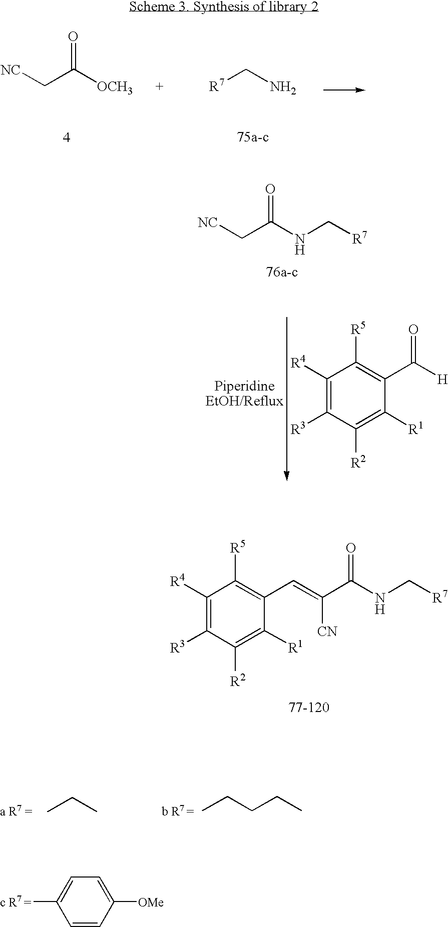

Surprisingly, screening of library 2 compounds failed to reveal any with dynamin inhibition ≦100 μM. This is more surprising given that the original screening data showed that tyrphostin A47 (2) displayed a dynamin IC₅₀=70 μM.

Closer examination of tyrphostin A47 afforded a potential explanation for the failure to detect inhibitory activity in library 2. That is, the single —S was potentially available for oxidation in solution to the corresponding dimeric structure. Simple tautomerisation followed by oxidation yields the corresponding disulfide species (121) (see Scheme 4). Freshly prepared solutions of 2 showed no inhibitory potency, while stocks kept at room temperature for 24 hrs showed weak potency. The IC₅₀ of 121 decreased to >300 μM when the reducing reagent dithiothreitol (2 mM) was included in the dynamin assay medium. Dithiothreitol alone was without effect on dynamin GTPase activity (data not shown). The in situ generation of the dimeric 121 affords a similar low energy conformation with the required key functional groups appropriately disposed to ensure good inhibition of dynamin. A similar sequence of events has been observed for thioindoles which are EGFR tyrosine kinase inhibitors which showed increased activity upon oxidation (Thompson et al., 1993). 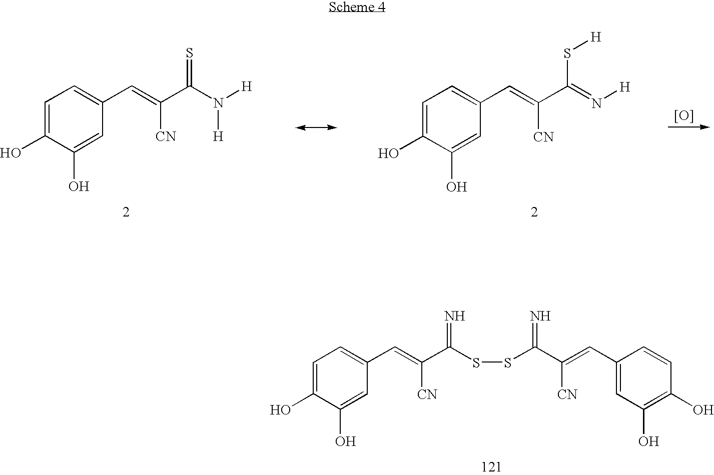
2.4 Discussion

The structure-activity relationship of dimeric typhostins against the GTPase enzyme dynamin was evaluated via the synthesis and screening of a library of compounds based upon the lead compounds bis-tyrphostin and typhostin A47. From the results obtained, potent inhibitory activity was found in dimeric tyrphostin compounds containing two aromatic rings with hydroxy groups in the 3,4 positions. Modifications to these compounds can be readily made by altering which functional groups are used to form the spacer.

### EXAMPLE 3

### Development of Prodrugs

Prodrugs of bis-tyrphostin and analogues thereof were developed to increase cell membrane permeability characteristics and thereby increase potency in cells. A suitable reaction for providing prodrugs of dimeric tyrphostin compounds is illustrated in Scheme 5. Bis-tyrphostin is exemplified as the starting reagent. The dimeric tyrphostin compound is stirred with appropriate anhydride or acid chloride (in molar excess) in a pyridine/N,N-dimethylformamide (DMF) solution in the presence of an appropriate catalyst such as dimethylaminopyridine (DMAP). In some cases, the solution may need to be refluxed to drive the reaction to completion. On completion of the reaction, the esterified product is purified by either recrystallization or by chromatography. Examples of prodrugs developed are shown in Table 2 and Table 3.

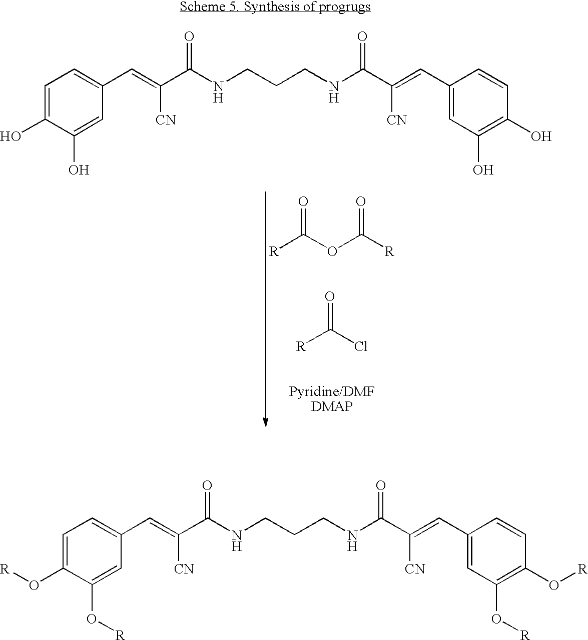

TABLE 2
Prodrugs of bis-tyrphostin.

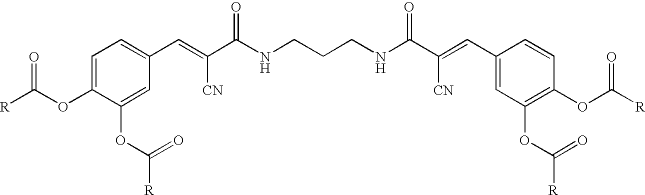

| Prodrug | R |
|---|---|
| TH-1 | CH₃ |
| TH-2 | 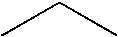 |
| TH-3 | 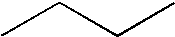 |
| TH-4 | 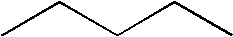 |
| TH-5 | 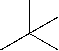 |
| TH-6 | 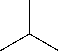 |
| TH-7 | 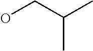 |
| TH-8 | 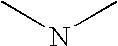 |
| TH-9 | 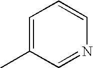 |

TABLE 3
Prodrug forms of a dimeric tyrphostin

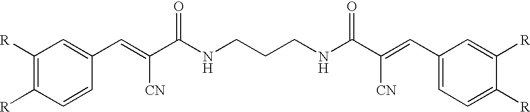

| Prodrug | R | Mw |
|---|---|---|
| Pro-BisT | 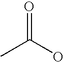 | 616.59 |
| 80-1 | 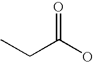 | 672.68 |
| 80-2 | 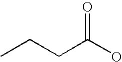 | 728.78 |
| 80-3 | 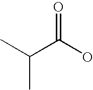 | 728.78 |
| 80-4 | 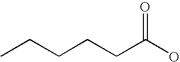 | 841.00 |
| 80-5 | 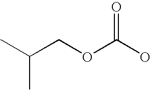 | 964.04 |
| 80-6 | 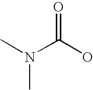 | 723.74 |
| 81-1 | 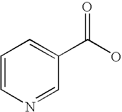 | 868.80 |
| 81-2 | 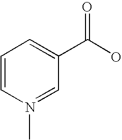 | 928.942 |

The prodrug Pro-BisT has 4 acetyl ester groups in place of the 4 hydroxyl groups present on bis-tyrphostin and was evaluated for capacity to cross the outer cellular membranes of cells. Pro-BisT is converted into the active compound bis-tyrphostin within cells, which is then able to bind to dynamin and thereby inhibit endocytosis. Pro-BisT was found to inhibit receptor-mediated endocytosis (RME) of transferrin or EGF in the cell lines Hela, HER14, COS7, Swiss 3T3, A431, B104 and B35. Pro-BisT is significantly more potent than bis-tyrphostin (˜30×) and efficiently blocks RME in the cell lines tested at concentrations of between 10-20 μM indicating greatly improved ability to penetrate cells compared to bis-tyrphostin.

The prodrug 80-1 was found to inhibit RME at similar concentrations to Pro-BisT (10-20 μM) and was developed to reduce premature hydrolysis of the prodrug in the external cellular environment prior to passage into cells. This prodrug has an improved shelf life when stored in powder form compared to Pro-BisT.

It will be appreciated by persons skilled in the art that numerous variations and/or modifications may be made to the invention as shown in the specific embodiments without departing from the spirit or scope of the invention as broadly described. The present embodiments are, therefore, to be considered in all respects as illustrative and not restrictive.

### REFERENCES

1. Aridor, M. & Hannan, L. A. (2000) Traffic jam: a compendium of human diseases that affect intracellular transport processes. Traffic 1, 836-851.
2. Aridor, M. & Hannan, L. A. (2002) Traffic jams II: an update of diseases of intracellular transport. Traffic 3, 781-790.
3. Atwood, W. J. (2001) A combination of low-dose chlorpromazine and neutralizing antibodies inhibits the spread of JC virus (JCV) in a tissue culture model: Implications for prophylactic and therapeutic treatment of progressive multifocal leukencephalopathy. J Neurovirol. 7, 307-310.
4. Berge, S M et al (1997) J. Pharmaceutical Sciences. 66:1-19.
5. Brodin, L., Low, P. & Shupliakov, O. (2000) Sequential steps in clathrin-mediated synaptic vesicle endocytosis. Curr. Opin. Neurobiol. 10, 312-320. Cataldo A. et al. J. Neurosci. 1997, 17:6142.
6. Butler M. H., David C., Ochoa G. C., Freyberg Z., Daniell L., Grabs D., Cremona O., and De Camilli P. (1997) Amphiphysin II (SH3P9; BIN1), a member of the amphiphysin/rvs family, is concentrated in the cortical cytomatrix of axon initial segments and nodes of ranvier in brain and around T tubules in skeletal muscle. J Cell Biol 137, 1355-1367.
7. Cataldo, A., et al., J. Neurosci. 1997, 17:6142
8. Cataldo, A., Rebeck, G. W., Ghetri, B., Hulette, C., Lippa, C., Van Broeckhoven, C., van Duijn, C., Cras, P., Bogdanovic, N., Bird, T., Peterhoff, C. & Nixon, R. (2001) Endocytic disturbances distinguish among subtypes of Alzheimer's disease and related disorders. Ann. Neurol. 50, 661-665.
9. Cousin M. A. and Robinson P. J. (2000a) Ca2+ inhibition of dynamin arrests synaptic veside recycling at the active zone. J. Neurosci. 20, 949-957.
10. Cousin M. A. and Robinson P. J. (2000b) Two mechanisms of synaptic vesicle recycling in rat brain nerve terminals. J. Neurochem. 75, 1645-1653.
11. Cousin M. A. and Robinson P. J. (2001) The dephosphins: Dephosphorylation by calcineurin triggers synaptic vesicle endocytosis. Trends Neurosci. 24, 659-665.
12. Di Paolo, G., Sankaranarayanan, S., Wenk, M. R., Daniell, L., Perucco, E., Caldarone, B. J., Flavell, R., Picciotto, M. R., Ryan, T. A., Cremona, O. & De Camilli, P. (2002) Decreased synaptic vesicde recycling efficiency and cognitive deficits in amphiphysin 1 knockout mice. Neuron 33, 789-804.
13. Duan, D., Li, Q., Kao, A. W., Yue, Y., Pessin, J. E. & Engelhardt, J. F. (1999) Dynamin is required for recombinant adeno-associated virus type 2 infection. J. Virol. 73, 10371-10376.
14. Dunkley P. R., Jarvie P. E., Heath J. W., Kidd G. J., and Rostas J. A. (1986) A rapid method for isolation of synaptosomes on Percoll gradients. Brain Res. 372, 115-129.
15. Gazit, A., Osherov, N., Gilon, C. & Levitzki, A. (1996) Tyrphostins. 6. Dimeric benzylidenemalononitrile tyrpphostins: patent inhibitors of EGF receptor tyrosine kinase in vitro. J. Med. Chem. 39, 4905-4911.
16. Gray, N. W., Fourgeaud, L., Huang, B., Chen, J., Cao, H., Oswald, B. J., Hemar, A. & McNiven, M. A. (2003) Dynamin 3 is a component of the postsynapse, where it interacts with mGluR5 and homer. Curr. Biol. 13, 510-515.
17. Grieb, T. A. & Burgess, W. H. (2000) The mitogenic activity of fibroblast growth factor-1 correlates with its internalization and limited proteolytic processing. J. Cell. Physiol. 184, 171-182.
18. Humeau, Y., Doussau, F., Grant, N. J. & Poulain, B. (2000) How botulinum and tetanus neurotoxins block neurotransmitter release. Biochimie 82, 427-446.
19. Koenig, J. H. & Ikeda, K. (1989) Disappearance and reformation of synaptic vesicle membrane upon transmitter release observed under reversible blockage of membrane retrieval. J. Neurosci. 9, 3844-3860. Marquez-Sterling N. et al. J. Neurosci. 1997, 17:140
20. Marks B. and McMahon H. T. (1998) Calcium triggers calcineurin-dependent synaptic vesicle recycling in mammalian nerve termiinals. Curr. Biol. 8, 740-749.
21. Marquez-Sterling, N. R., Lo, A. C., Sisodia, S. S. & Koo, E. H. (1997) Trafficking of cell-surface b-amyloid precursor protein: Evidence that a sorting intermediate participates in synaptic veside recycling. J. Neurosci. 17, 140-151.
22. McCluskey, A., Hill, T. A., Robinson, P. J., Scott, J. L. & Edwards, J. K. (2002) Green chemistry approaches to the Knoevenagel condensation: Comparison of ethanol, water and solvent free (dry grind) approaches. Tetrahedron Lett. 43, 3117-3120.
23. Metzler, M., Legendre-Guillemin, V., Gan, L., Chopra, V., Kwok, A., McPherson, P. S. & Hayden, M. R. (2001) HIP1 functions in clathrin-mediated endocytosis through binding to dathrin and AP2. J. Biol. Chem. 276, 39271-39276.
24. Niemann, H. H., Knetsch, M. L., Scherer, A., Manstein, D. J. & Kull, F. J. (2001) Crystal structure of a dynamin GTPase domain in both nucleotide-free and GDP-bound forms. EMBO J. 20, 5813-5821.
25. Ong, W. Y., Kumar, U., Switzer, R. C., Sidhu, A., Suresh, G., Hu, C. Y. & Patel, S. C. (2001) Neurodegeneration in Niemann-Pick type C disease mice. Exp. Brain Res. 141, 218-231.
26. Remington: The Science and Practice of Pharmacy (Mack Publishing Co., 1995).
27. Robinson P. J., Sontag J.-M., Liu J. P., Fykse E. M., Slaughter C., McMahon H. T., and Südhof T. C. (1993) Dynamin GTPase regulated by protein kinase C phosphorylation in nerve terminals. Nature. 365, 163-166.
28. Roy, A. M., Parker, J. S., Parrish, C. R. & Whittaker, G. R. (2000) Early stages of influenza virus entry into Mv-1 lung cells: involvement of dynamin. Virology. 267, 17-28.
29. Schwake, M., Friedrich, T. & Jentsch, T. J. (2001) An internalization signal in ClC-5, an endosomal Cl-channel mutated in Dent's disease. J. Biol. Chem. 276, 12049-12054.
30. Seto, E. S., Bellen, H. J. & Lloyd, T. E. (2002) When cell biology meets development: endocytic regulation of signaling pathways. Genes Dev. 16, 1314-1336.
31. Sever, S., Muhlberg, A. B. & Schmid, S. L. (1999) Impairment of dynamin's GAP domain stimulates receptor-mediated endocytosis. Nature. 398, 481-486
32. Smith, D. S., Niethammer, M., Ayala, R., Zhou, Y., Gambello, M. J., Wynshaw-Boris, A. & Tsai, L. H. (2000) Regulation of cytoplasmic dynein behaviour and microtubule organization by mammalian Lis1. Nat. Cell Biol. 2, 767-775.
33. Smythies, J. (2000) What is the function of receptor and membrane endocytosis at the postsynaptic neuron? Proc. R. Soc. Lond. Biol. Sci. 267, 1363-1367.
34. Stowell, M. H., Marks, B., Wigge, P. & McMahon, H. T. (1999) Nucleotide-dependent conformational changes in dynamin: evidence for a mechanochemical molecular spring. Nat. Cell Biol. 1, 27-32.
35. Sung, J. Y., Kim, J., Paik, S. R., Park, J. H., Ahn, Y. S. & Chung, K. C. (2001) Induction of neuronal cell death by Rab5A-dependent endocytosis of alpha-synuclein. J. Biol. Chem. 276, 27441-27448.
36. Tan, T. C., Valova, V. A., Malladi, C. S., Graham, M. E., Berven, L. A., Jupp, O. J., Hansra, G., McClure, S. J., Sarcevic, B., Boadle, R. A., Larsen, M. R., Cousin, M. A. & Robinson, P. J. (2003) Cdk5 is essential for synaptic vesicle endocytosis. Nat. Cell Biol. 5, 701-710.
37. Thompson, M.; Rewcastle, G. W.; Tercel, M.; Dobrusin, E. M.; Fry, D. W.; Kraker A. J.; Denny, W. A. Tyrosine Kinase Inhibitors. 1. Structure-Activity Relationships for Inhibition of Epidermal Growth Factor Receptor Tyrosine Kinase Activity by 2,3-Dihydro-2-thioxo-1H-indole-3-alkanoic Acids and 2,2′-Dithiobis(1H-indole-3-alkanoic acids). J. Med. Chem. 1993, 36, 2459-2469.
38. van der Bliek A. M., Redelmeier T. E., Dam ke H., Tisdale E. J., Meyerowitz E. M., and Schmid S. L. (1993) Mutations in human dynamin block an intermediate stage in coated vesicle formation. J. Cell Biol. 122, 553-563.
39. Wang, L.-H., Rothberg, K. G. & Anderson, R. G. (1993) Mis-assembly of clathrin lattices on endosomes reveals a regulatory switch for coated pit formation. J. Cell Biol. 123, 1107-1117.
40. Wyss, S., Berlioz-Torrent, C., Boge, M., Blot, G., Honing, S., Benarous, R. & Thali, M. (2001) The highly conserved C-terminal dileucine motif in the cytosolic domain of the human immunodeficiency virus Type 1 envelope glycoprotein is critical for its association with the AP-1 clathrin adapter. J. Virol. 75, 2982-2992.

1. A method of inhibiting dynamin-dependent endocytosis in cells, the method comprising treating the cells with an effective amount of a compound of formula I, or a physiologically acceptable salt thereof, wherein
M-Sp-M′ Formula I
M and M′ are each independently a moiety of formula II and are the same or different, and Sp is a spacer; 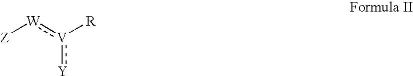
V is C or CH;
W is CH or a linker group; and
Y is hydrogen, cyano, nitro, NH, amino, oxo, halo, hydroxy, sulfhydryl, carboxy, thiocarboxy, sulfur, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur; or
W, V and Y form a 5 or 6 membered substituted or unsubstituted heterocyclic or carbocyclic ring fused with Z, wherein the heterocyclic ring includes from 1 to 3 heteroatoms selected from O, N and S, and the heterocyclic or carbocyclic ring, when substituted, has at least one substituent selected from cyano, nitro, NH, amino, oxo, halo, hydroxy, sulfhydryl, carboxy, thiocarboxy, sulfur, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur; and
R is CH₂R′, CXR′ or CHX′R′;
X is O or S;
X′ is cyano, nitro, amino, halo, hydroxy, sulfhydryl, carboxy, thiocarboxy, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur;
R′ is NH, O or S bonded to the spacer; and
Z is selected from:
(a) an unsubstituted heterocyclic group consisting of one or two rings independently having 5 or 6 ring members including up to 3 heteroatoms selected from O, N and S;
(b) an unsubstituted carbocyclic group consisting of one or two rings independently having 5 or 6 ring members;
(c) a heterocyclic group consisting of one or two rings independently having 5 or 6 ring members including up to 3 heteroatoms selected from O, N and S wherein the heterocyclic group has one or more substituents independently selected from:
(i) nitro, NH, amino, cyano, halo, hydroxy, carboxy, oxo, sulfur, sulfhydryl, C₁-C₂ alkoxy and C₁-C₂ acyl; and
(ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl; and
(d) a carbocyclic group consisting of one or two rings independently having 5 or 6 ring members, and at least two substituents when W is CH or a linker group or W, V and Y form an unsubstituted carbocyclic group, or at least one substituent when W, V and Y form a heterocyclic group, independently selected from:
(i) nitro, NH, amino, cyano, halo, hydroxy, carboxy, oxo, sulfur, sulfhydryl, C₁-C₂ alkoxy and C₁-C₂ acyl; and
(ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl;
wherein when Z of one of M or M′ is selected from (b), Z of the other of M or M′ is selected from (a), (c) or (d).

2-25. (canceled)

26. A method of prophylaxis or therapeutic treatment of a disease or condition in a mammal mediated by dynamin-dependent endocytosis, the method comprising administering to the mammal an effective amount of a compound of Formula I according to claim 1, or a physiologically acceptable salt, or prodrug thereof.

27-52. (canceled)

53. A method for identifying a dimeric tyrphostin or an analogue thereof with ability to inhibit GTPase activity of dynamin, the method comprising:
incubating the dimeric tyrphostin or analogue thereof with dynamin or a molecule having dynamin GTPase activity to provide test data; and
determining whether the dimeric tyrphostin or analogue thereof inhibits the GTPase activity of dynamin on the basis of the test data.

54. (canceled)

55. A compound of Formula III or a physiologically acceptable salt, or prodrug thereof, wherein:
M-Sp-M′ Formula III
M and M′ are each independently a moiety of formula II and are the same or different, and Sp is a spacer; 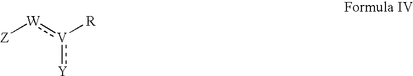
V is C or CH;
W is CH or a linker group; and
Y is hydrogen, cyano, nitro, NH, amino, oxo, halo, hydroxy, sulfhydryl, carboxy, thiocarboxy, sulfur, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur; or
W, V and Y form a 5 or 6 membered substituted or unsubstituted heterocyclic or carbocyclic ring fused with Z, wherein the heterocyclic ring includes from 1 to 3 heteroatoms selected from O, N and S, and the heterocyclic or carbocyclic ring, when substituted, has at least one substituent selected from cyano, nitro, NH, amino, oxo, halo, hydroxy, sulfhydryl, carboxy, thiocarboxy, sulfur, or an unsubstituted C₁-C₃ group or a C₁-C₃ group substituted with at least one group independently selected from cyano, nitro, NH, amino, oxo, halo, hydroxy, sulfhydryl, carboxy, thiocarboxy and sulfur; and
R is CH₂R′, CXR′ or CHX′R′;
X is O or S;
X′ is cyano, nitro, amino, halo, hydroxy, sulfhydryl, carboxy, thiocarboxy, or an unsubstituted C₁-C₃ group or C₁-C₃ group substituted with at least one group independently selected from cyano, nitro, NH, amino, oxo, halo, hydroxy, sulfhydryl, carboxy, thiocarboxy and sulfur;
R′ is NH, O or S bonded to the spacer; and
Z is selected from:
(a) an unsubstituted heterocyclic group consisting of one or two rings independently having 5 or 6 ring members including up to 3 heteroatoms selected from O, N and S;
(b) an unsubstituted carbocyclic group consisting of one or two rings independently having 5 or 6 ring members;
(c) a heterocyclic group consisting of one or two rings independently having 5 or 6 ring members including up to 3 heteroatoms selected from O, N and S wherein the heterocyclic group has one or more substituents independently selected from:
(i) nitro, NH, amino, cyano, halo, hydroxy, carboxy, oxo, sulfur, sulfhydryl, C₁-C₂ alkoxy and C₁-C₂ acyl; and
(ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl; and
(d) a carbocyclic group consisting of one or two rings independently having 5 or 6 ring members, and at least two substituents when W is CH or a linker group or W, V and Y form an unsubstituted carbocyclic group, or at least one substituent when W, V and Y form a heterocyclic group, independently selected from:
(i) nitro, NH, amino, cyano, halo, hydroxy, carboxy, oxo, sulfur, sulfhydryl, C₁-C₂ alkoxy and C₁-C₂ acyl; and
(ii) a C₁-C₂ alkyl or C₁-C₂ alkenyl group with at least one substituent selected from nitro, NH, amino, cyano, halo, hydroxy, carboxy, oxo, sulfur, sulfhydryl, C₁-C₂ alkoxy and C₁-C₂ acyl;
wherein when Z of one of M or M′ is selected from (b), Z of the other of M or M′ is selected from (a), (c) or (d), and with the proviso that Z of at least one of M and M′ is other than a benzyl group of formula IVa when R is CXR′, X is O, R′ is NH bonded to the spacer, V is C, W is CH, Y is cyano, and 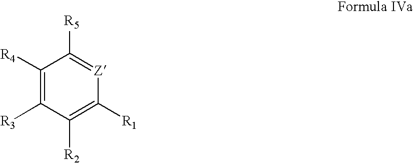
R₁, R₂, and R₅ are H, and R₃ and R₅ are hydroxy; or
R₁ and R₅ are H, and R₂ to R₄ are hydroxy when Sp is a C₂-C₄ alkyl spacer; and
wherein Z′ is a carbon atom bonded to W.

56. A compound according to claim 55 wherein:
V is C;
W is CH;
Y is hydrogen, cyano, nitro, amino, halo, hydroxy, sulfhydryl, carboxy, thiocarboxy, or an unsubstituted C₁-C₂ group or C₁-C₂ group substituted with at least one group independently selected from cyano, nitro, NH, amino, oxo, halo, hydroxy, sulfhydryl, carboxy, thiocarboxy and sulfur; or
W, V and Y form a 5 or 6 membered substituted or unsubstituted heterocyclic or carbocyclic ring fused with Z, wherein the heterocyclic ring includes from 1 to 3 heteroatoms selected from O, N and S, and the carbocyclic or heterocyclic ring, when substituted, has at least one substituent selected from cyano, nitro, NH, amino, oxo, halo, hydroxy, sulfhydryl, carboxy, thiocarboxy and sulfur, or an unsubstituted C₁-C₂ group or C₁-C₂ group substituted with at least one group independently selected from cyano, nitro, NH, amino, oxo, halo, hydroxy, sulfhydryl, carboxy, thiocarboxy and sulfur; and
R is CH₂R′, CXR′ or CHX′R′;
X is O or S; and
X′ is cyano, nitro, amino, halo, hydroxy, sulfhydryl, carboxy, thiocarboxy, or an unsubstituted C₁-C₂ group or C₁-C₂ group substituted with at least one group independently selected from cyano, nitro, NH, amino, oxo, halo, hydroxy, sulfhydryl; carboxy, thiocarboxy and sulphur.

57. A compound according to claim 56 wherein:
Y is cyano, nitro, amino, carboxy, hydroxy, sulfhydryl, thiocarboxy, or a C₁-C₂ group substituted with a group selected from cyano, nitro, amino, hydroxy, sulfhydryl, carboxy and thiocarboxy;
W, V and Y form a 5 or 6 membered substituted or unsubstituted heterocyclic or carboxylic ring fused with Z, wherein the heterocyclic ring includes from 1 to 3 heteroatoms selected from O, N and S, and the carbocyclic or heterocyclic ring, when substituted, has at least one substituent selected from cyano, nitro, amino, hydroxy, sulfhydryl, carboxy and thiocarboxy, or a C₁-C₂ group substituted with a group selected from cyano, nitro, amino, hydroxy, sulfhydryl, carboxy and thiocarboxy; and
R is CXR′.

58. A compound according to claim 55 wherein Z is selected from:
(i) a heterocyclic group consisting of one or two rings independently having 5 or 6 ring members including up to 3 heteroatoms independently selected from O, N and S;
(ii) a heterocyclic group consisting of one or two rings independently having 5 or 6 ring members including up to 3 heteroatoms independently selected from O, N and S, wherein the heterocyclic group has one or more substituents independently selected from nitro, NH, halo, cyano, amino, hydroxy, carboxy, oxo, sulfur, and C₁-C₂ alkoxy; and
(iii) an carbocyclic group consisting of one or two rings independently having 5 or 6 ring members, and at least two substituents independently selected from nitro, NH, amino, halo, cyano, hydroxy, carboxy, oxo, sulfur and C₁-C₂ alkoxy.

59. A compound according to claim 55 wherein Z of at least one of M and M′ is other than a 2,3-disubstituted carboxycyclic group.

60. A compound according to claim 55 wherein Z of at least one of M and M′ comprises:
at least two substituents in ortho positions relative to one another or in adjacent substitution positions, when the Z group is selected from (d) and W is CH or a C₁-C₃ linker group; or
the, or one of, the substituents on a carbon atom adjacent to the, or one of the, heteroatom (s) when the Z group is a heterocyclic group selected from (c); or
when W, V and Y are cyclised forming a heterocyclic ring fused with Z, the, or one of, the substituents on a carbon atom spaced at least one bond length from the heterocyclic ring.

61. A compound according to claim 55 wherein when the Y substituent of one of M or M′ is hydrogen, the Y substituent of the other of M and M′ is other than hydrogen.

62. A compound according to claim 55 wherein W, V and Y form a 5 or 6 membered heterocyclic ring fused with Z.

63. A compound according to claim 62 wherein the heterocyclic ring fused with Z forms a two ring heterocyclic group.

64. A compound according to claim 55 wherein Z comprises an aryl group consisting of one or two rings independently having 5 or 6 ring members, and at least two substituents independently selected from nitro, NH, amino, halo, cyano, hydroxy, carboxy, oxo, sulphur and C₁-C₂ alkoxy.

65. A compound according to claim 64 wherein Z comprises an aryl group consisting of one ring having 6 ring members and at least two substituents independently selected from nitro, amino, halo, cyano, hydroxy, carboxy and C₁-C₂ alkoxy.

66. A compound according to claim 65 wherein the aryl group has at least two substituents independently selected from nitro, amino, and hydroxy.

67. A compound according to claim 55 wherein Z comprises a heterocyclic group having one or two rings independently having 5 or 6 ring members including up to 3 heteroatoms selected from O, N and S, wherein the heterocyclic group has one or more substituents independently selected from nitro, NH, halo, cyano, amino, hydroxy, carboxy, oxo, sulphur and C₁-C₂ alkoxy.

68. A compound according to claim 67 wherein the heterocyclic group has one or more substituents independently selected from nitro, amino and hydroxy.

69. A compound according to claims 55 wherein M and M′ are each independently a moiety as follows: 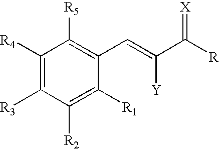
wherein: X is O or S;
Y is cyano, nitro, amino, halo, hydroxy, sulfhydryl, carboxy, or thiocarboxy; or
R₁ and Y are cyclised forming a 5 or 6 membered substituted or unsubstituted heterocyclic or carbocyclic ring, wherein the heterocyclic ring includes 1 or 2 heteroatoms selected from O, N and S, and the carbocyclic or heterocyclic ring, when substituted, has at least one substituent selected from cyano, nitro, NH, amino, oxo, halo, hydroxy, sulfhydryl, carboxy, thiocarboxy and sulfur; and R₂ to R₅ are independently hydrogen or a substituent independently selected from nitro, amino, halo, hydroxy, carboxy, sulfhydryl, thiocarboxy, C₁-C₂ alkoxy and C₁-C₂ acyl; or
R₂ to R₅ are independently hydrogen or a substituent independently selected from nitro, amino, halo, hydroxy, carboxy, sulfhydryl, thiocarboxy, halo, C₁-C₂ alkoxy and C₁-C₂ acyl; and
R₁ to R₅ are independently hydrogen or a substituent independently selected from nitro, amino, halo, hydroxy, carboxy, sulfhydryl, thiocarboxy, halo, C₁-C₂ alkoxy and C₁-C₂ acyl; and
R is NH, O is S bonded to the spacer Sp; and
wherein at least one of M and M′ is characterised in that, at least two of R₁ to R₅, are other than hydrogen and when R₁ to R₂ are other than hydrogen at least one of R₃ to R₅ is also other than hydrogen, or when R₁ and Y are cyclised, at least two of R₂ to R₅ are other than hydrogen when R₁ and Y form an unsubstituted carbocyclic group or at least one of R₂ to R₅ is other than hydrogen when Y and R₁ form a heterocyclic group.

70. A compound according claim 69 wherein R₁ to R₃ are other than hydrogen.

71. A compound according to claim 69 wherein at least two of R₁ to R₅ are in ortho positions relative to one another.

72. A compound according to claim 69 wherein at least one of M and M′ has three substituents and wherein the substituents are adjacent to one another.

73. A compound according to claim 72 wherein either R₁ to R₃ are other then hydrogen or R₂ to R₅ are other than hydrogen.

74. A compound according to claim 69 wherein when at least one of R₁ to R₅ or R₂ to R₅ is halo, C₁-C₂ alkoxy or C₁-C₂ acyl, at least one other substituent is selected from nitro, amino, hydroxy, carboxy and thiocarboxy when R₁ and Y are cyclised and form a heterocyclic ring, or at least two other substituents are selected from nitro, amino, hydroxy, carboxy and thiocarboxy when R₁ and Y are not cyclised or form an unsubstituted carboxylic ring.

75. A compound according to claim 69 wherein Y is cyano, X is O and R is NH.

76. A compound according to claim 55 wherein M and M′ are the same.

77. A compound according to claim 55 wherein the spacer Sp permits the compound to adopt a hairpin conformation.

78. A compound according to claim 55 wherein the spacer Sp comprises an unsubstituted alkane chain as follows:
—(CH₂)nCH₂—
wherein n is an integer of from 1 to 5.

79. A compound according to claim 1 wherein the compound of Formula I is a dimeric tyrphostin.

80. A pharmaceutical composition comprising a compound as defined in claim 55 together with a physiologically acceptable excipient, carrier or diluent.
